(19) United States
(12) Patent Application Publication
(10) Pub. No.: US 2024/0409612 A1
(43) Pub. Date: Dec. 12, 2024

(54) IMPROVED PURIFICATION PROCESS

(71) Applicant: CSL BEHRING AG, Bern (CH)

(72) Inventors: Ibrahim EL MENYAWI, Bern (CH); Simon Stephan GERBER, Worben (CH); Priskus KIPFER, Brugg (CH)

(73) Assignee: CSL BEHRING AG, Bern (CH)

(21) Appl. No.: 18/697,387

(22) PCT Filed: Sep. 30, 2022
(86) PCT No.: PCT/EP2022/077382

§ 371 Date: Mar. 29, 2024

(30) Foreign Application Priority Data
Oct. 1, 2021 (EP) 21200554.0

(51) Int. Cl. C07K 14/765; C07K 1/18

(52) U.S. Cl. CPC C07K 14/765; C07K 1/18

(57) ABSTRACT

The present invention relates to improved industrial scale processes for removing metal cations, such as copper, from blood plasma derived albumin products and pharmaceutical compositions derived therefrom. The albumin sample is contacted with a ligand for binding to or sequestering the metal cations, said ligand is a chelating agent, most preferably a biodegradable chelating agent, or a chelating resin, most preferably a cation-exchange chelating resin.

### FIELD OF THE INVENTION

The present invention relates to improved industrial scale processes for purifying metal cations, such as copper, from blood plasma derived albumin and pharmaceutical compositions derived therefrom.

### RELATED APPLICATION

This application claims priority from European patent application EP 21200554.0, the entire contents of which are hereby incorporated by reference.

### BACKGROUND OF THE INVENTION

Serum albumin is one of the most abundant proteins present in blood (35-55 g/L; approximately 60% of total protein). It functions within the blood as a carrier of hydrophobic molecules such as fatty acids and as a scavenger that binds to a range of molecules including organic molecules and cations (such as Ca2+, Na+ and K+), sequestering such molecules until they can be eliminated. Because of albumin's abundance, small relative size (66.5 kDa) and high charge at physiological pH its main function is to regulate the oncotic pressure of blood so as to maintain fluid in the bloodstream and prevent leakage into surrounding tissues

During World War II, it was recognized that human serum albumin (hSA) can be formulated in a physiologically appropriate solution useful for replacing blood volume lost due to trauma or surgery (e.g., Cohn et al. (1946), J. Amer. Chem. Soc. 68:459-75). Today, hSA is routinely administered in clinical settings when there are existing or anticipated clinical problems or complications from reduced oncotic pressure, and/or as an adjunct to diuretic therapy. For example, hSA may be used for the resuscitation of patients in shock due to acute loss of blood or plasma. Extensive burns are often followed by sequential shifts in the distribution of body water, salt and proteins resulting in hypovolaemic shock and circulatory failure. Thus, administration of hSA has been found to assist recovery of burn victims. In another example, in patients who have undergone abdominal surgery, the intravenous (IV) administration of albumin solution (20%) immediately after the operation has been shown to improve lung compliance and gaseous exchange.

Different processes for preparing injectable hSA solutions have been described. Albumin's unique physiochemical properties allow relatively effective purification by precipitation methods. Albumin has the highest solubility and the lowest isoelectric point (the pH at which it bears no net charge) of the major plasma proteins. Adjustments to pH, temperature, ionic strength, ethanol or salt concentration and protein concentration therefore allow the fractionation of albumin from the other plasma proteins. These fractionation methods include precipitation with cold ethanol, using methods such as Cohn or Kistler and Nitschmann for industrial scale manufacture of pharmaceutical grade hSA.

These industrial scaled cold ethanol fractionation methods enable multiple plasma proteins to be extracted from the one plasma source. Such processes generally involve frozen plasma (batch sizes in the range of 1000-15000 kg) being thawed to form an albumin rich cryosupernatant and a cryoprecipitate. The cryoprecipitate contains valuable coagulation factors that are subsequently separated from the cryosupernatant. In the Cohn or Kistler and Nitschmann processes, the cryosupernatant may be optionally exposed to an initial low ethanol (typically 8%) precipitation stage to remove Fibrinogen. Again, the precipitate (Fraction I) is removed and can be used to make other products such as Fibrinogen. Adsorption steps using ion-exchange resins or affinity are also optionally conducted across either of these two intermediate fractions to extract other proteins (e.g. Prothrombin complex; Antithrombin III; C1 esterase inhibitor). Subsequently, the albumin is extracted from the Supernatant I by raising the ethanol concentration to about 25% at about pH 6.9 for the Cohn method or about 19% at about pH 5.85 for the Kistler and Nitschmann method, the immunoglobulins are precipitated (Fraction (I+)II+III or Precipitate A) while the albumin remains in solution (Supernatant (I+)II+III or Filtrate A). The hSA is then isolated from the majority of the other plasma contaminants (mainly α and β globulins), which are precipitated by the further addition of ethanol to a final ethanol concentration of about 40% (Fraction IV). In a final step, the albumin is itself precipitated near its isoelectric point. The precipitate paste (Fraction V) can be held frozen before further processing. It is important to recognise that these processes have some adaptability and have been optimised over the years to suit each manufacturers product portfolio. An example of this would be the presence or absence of an additional Cohn fractionation step (Fr IV₁) following Fr (I+)II+II step that can be used to extract alpha-1-antitrypsin.

An alternative to cold ethanol fractionation is chromatographic purification of plasma to produce albumin. This method was first described in the early 1980s. After clarification, the plasma is buffer-exchanged by either column gel filtration or diafiltration to allow subsequent ion exchange chromatography. There follows one or more column chromatographic purification steps, then further gel filtration chromatography or buffer exchange. Such methods have not however been widely adopted due mainly to the scale of plasma manufacture and hence the size, complexity and cost of the chromatographic equipment. Combined methods, whereby chromatographic purification steps supplement the cold ethanol fractionation process, have been adopted by a number of manufacturers including CSL (e.g. Albumex® process). Single or multiple column steps can improve product purity by allowing convenient buffer exchange and depleting trace protein contaminants.

Several other strategies for the purification of albumin have been evaluated over the years including salt precipitation methods, such as those described in U.S. Pat. No. 11,028,125. To date none of these methods have been developed into full scale commercial processes for making hSA suitable for pharmaceutical use.

Recombinant production of hSA in transgenic animals is appealing because of the large amount of protein that can be quickly obtained, thereby making it possible to produce significant quantities. However, such methods are costly and time consuming. Consequently, hSA utilised in the clinic is generally obtained by fractionation of pooled donor blood derived plasma.

hSA manufacture is completed by solubilising the albumin, formulating with stabilisers (sodium caprylate and/or acetyl tryptophanate) and pasteurisation. The pasteurisation process (60° C. for 10 hours) was introduced for albumin pharmaceutical products in the 1940s. The step inactivates lipid and non-lipid enveloped viruses including hepatitis A, B and C and HIV. The inclusion of the stabilisers ensures that the albumin solution is not denatured on heating. Most regulatory agencies require the step to be conducted in the final container, although terminal bulk pasteurisation has been accepted by a few regulatory agencies, such as the Therapeutic Goods Administration in Australia.

Pharmaceutical hSA compositions are produced at two concentrations. The 4-5% hSA solution is an isotonic solution particularly suitable for fluid replacement in hypovolaemia. The 20-25% hSA is a hypotonic but hyperoncotic solution for the treatment of fluid loss where electrolyte or fluid load is contraindicated. The highly concentrated protein solution provides colloidal pressure while minimizing the additional salts and fluid volume that are infused.

The ideal pharmaceutical hSA product for these purposes would be monomeric albumin of very high purity, free from contamination with other plasma proteins, endotoxins, metal ions, albumin aggregates and prekallikrein activator (PKA). However, these purification processes do not ensure the complete removal of contaminants, some of which may impact on the integrity of the final product and potentially influence the tolerability of hSA infusion.

There is therefore a need for improved processes and methods for the purification of albumin obtained from blood sources.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

The present invention provides a process for reducing the level of metal cations in an albumin-comprising sample obtained from blood-derived plasma, the process comprising:
i) providing an albumin-comprising sample obtained from blood derived-plasma;
ii) contacting the albumin-comprising sample with a ligand for binding to or for sequestering metal cations to obtain a metal cation-depleted albumin-comprising sample;
iii) recovering the metal cation-depleted albumin-comprising sample.

The present invention provides a process for preparing a purified albumin composition from an albumin-comprising sample obtained from blood derived-plasma, the process comprising:
i) providing an albumin-comprising sample obtained from blood-derived plasma;
ii) contacting the albumin-comprising sample with a ligand for binding to or for sequestering metal cations to obtain a metal cation-depleted albumin-comprising sample;
thereby preparing a purified albumin composition.

The ligand for binding to or for sequestering metal cations may be a chelating agent, or a cation exchange resin.

Preferably, the ligand for binding to or for sequestering metal cations is a chelating agent. Accordingly, in a first aspect, the present invention provides a process for preparing a purified albumin composition from an albumin-comprising sample obtained from blood-derived plasma, the process comprising:
i) providing an albumin-comprising sample obtained from blood-derived plasma;
ii) contacting the albumin-comprising sample with a chelating agent to obtain a metal cation-depleted albumin-comprising sample;
thereby preparing a purified albumin composition.

The chelating agent may be any agent which is capable of binding to and sequestering metal ions. In an embodiment, the chelating agent binds to a divalent metal ion. In a preferred embodiment, the chelating agent binds to copper (II) ions, where the copper is optionally present in the albumin solution as copper (II) sulphide.

Accordingly, in particularly preferred embodiments of the first aspect of the invention, there is provided a process for removing copper (cupric) ions from an albumin-comprising sample obtained from blood-derived plasma, wherein the process comprises:
i) providing an albumin-comprising sample obtained from blood;
ii) contacting the albumin-comprising sample with a chelating agent for binding to cupric ions in order to obtain an albumin-comprising sample that is depleted of cupric ions;
thereby preparing a purified albumin composition.

In any embodiment, the chelating agent may be selected from the group consisting of: ethylenediaminetetraacetic acid (EDTA), ethylene glycol-bis(β-aminoethyl ether)-N,N,N′,N′-tetraacetic acid (EGTA), ethylenediamine-N,N′-disuccinic acid (EDDS), iminodisuccinic acid (IDS), methylglycinediacetic acid (MGDA), triethylenetetramine (Trien), iminodiacetic acid (IDA), nitrilotriacetic acid (NTA), tripolyphosphate (TPP), diethylenetriaminepentaacetic acid (DTPA), sodium diethyldithiocarbamate (DDC), L-Glutamic acid N,N-diacetic acid, tetrasodium salt (GLDA), and penicillamine, of any salt thereof, including a calcium or sodium salt thereof.

In an embodiment the chelating agent is a biodegradable chelating agent.

In a particularly preferred embodiments, the biodegradable chelating agent is selected from the group consisting of: ethylenediamine-N,N′-disuccinic acid (EDDS), iminodisuccinic acid (IDS), methylglycinediacetic acid (MGDA), and nitrilotriacetic acid (NTA), most preferably EDDS, or a salt thereof, including a calcium or sodium salt thereof.

In a preferred embodiment the biodegradable chelating agent is (S,S)-EDDS. Most preferably, the biodegradable chelating agent is the tri-sodium salt of (S,S)-EDDS.

The chelating agent may be provided at any concentration that is suitable for depleting metal cations from the albumin-comprising sample, optionally wherein the chelating agent is contacted with the albumin-comprising sample at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the chelating agent is contacted with the albumin-comprising sample at a concentration of from about 2.5 mM to about 100 mM, preferably from about 5 mM to about 50 mM, or between about 10 mM to about 25 mM, more preferably from about 15 mM to about 20 mM.

In certain embodiments, the concentration of albumin in the albumin-comprising sample is from about 40 g/L to about 250 g/L, and wherein the sample is contacted with chelator at a concentration of from about 10 μM to about 100 mM, preferably from about μM to about 15 mM, more preferably from about 50 μM to about 1 mM, most preferably from about 50 μM to about 500 μM, especially about 50 μM. In alternative embodiments, the concentration of chelator is from about 2.5 mM to about 100 mM, preferably from about 5 mM to about 50 mM, or between about 10 mM to about 25 mM, more preferably from about 15 mM to about 20 mM.

In certain embodiments, the concentration of albumin in the albumin-comprising sample is from about 50 g/L to about 200 g/L, or from about 100 g/L to about 170 g/L, preferably from about 135 g/L to about 155 g/L, more preferably about 140 g/L to about 150 g/L, and wherein the sample is contacted with chelator at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the concentration of the chelating agent is from about 2.5 mM to about 100 mM, preferably from about 5 mM to about 50 mM, or between about 10 mM to about 25 mM, more preferably from about 15 mM to about 20 mM.

In certain embodiments, the concentration of chelating agent is preferably less than 100 mM chelator, more preferably less than 50 mM chelator, or less than 25 mM chelator, or less than 15 mM chelator, or less than 5 mM chelator. Most preferably, the concentration of chelator is less than 1 mM or less than 500 μM, or less than 250 μM or less than 100 μM or 50 μM or less.

In certain embodiments, the concentration of chelating agent is calculated relative to the concentration of copper in the albumin-comprising sample. As such, in certain embodiments, the chelating agent is contacted with the albumin-comprising sample at an amount that corresponds to approximately at least a 2-fold higher concentration of chelator to copper, at least a 5-fold higher concentration of chelator to copper, at least 10-fold higher concentration of chelator to copper, at least 15-fold higher concentration of chelator to copper, at least 20-fold higher concentration of chelator to copper, at least a 25-fold higher concentration of chelator to copper, at least at least a 50-fold higher concentration of chelator to copper, at least a 75-fold higher concentration of chelator to copper, at least a 100-fold higher concentration of chelator to copper, at least a 125-fold higher concentration of chelator to copper, at least a 150-fold higher concentration of chelator to copper, at least a 175-fold higher concentration of chelator to copper, or at least a 200-fold higher concentration of chelator to copper.

In a preferred embodiment the biodegradable chelating agent is (S,S)-EDDS, optionally the tri-sodium salt thereof, and wherein the (S,S)-EDDS is contacted with the albumin-comprising sample at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the (S,S)-EDDS is contacted with the albumin-comprising sample at a concentration of from about 10 mM to about 20 mM, preferably about 15 mM.

In certain embodiments, the biodegradable chelating agent is (S,S)-EDDS, optionally the tri-sodium salt thereof, and wherein the (S,S)-EDDS concentration is less than 100 mM, preferably less than 50 mM, or less than 25 mM, or less than 15 mM, or less than 5 mM. Most preferably, the concentration of (S,S)-EDDS is less than 1 mM or less than 500 μM, or less than 250 μM or less than 100 μM or 50 μM or less

In certain embodiments, the concentration of (S,S)-EDDS to be used is calculated relative to the concentration of copper in the albumin-comprising sample. As such, in certain embodiments, the (S,S)-EDDS is contacted with the albumin-comprising sample at an amount that corresponds to approximately at least a 2-fold higher concentration of (S,S)-EDDS to copper, at least a 5-fold higher concentration of (S,S)-EDDS to copper, at least a 10-fold higher concentration of (S,S)-EDDS to copper, at least a 15-fold higher concentration of (S,S)-EDDS to copper, at least a 20-fold higher concentration of (S,S)-EDDS to copper, at least a 25-fold higher concentration of (S,S)-EDDS to copper or at least at least a 50-fold higher concentration of (S,S)-EDDS to copper, at least a 75-fold higher concentration of (S,S)-EDDS to copper, at least a 100-fold higher concentration of (S,S)-EDDS to copper, at least a 125-fold higher concentration of (S,S)-EDDS to copper, at least a 150-fold higher concentration of (S,S)-EDDS to copper, at least a 175-fold higher concentration of (S,S)-EDDS to copper, or at least a 200-fold higher concentration of (S,S)-EDDS to copper, or higher.

In certain embodiments, the concentration of albumin in the albumin-comprising sample is from about 40 g/L to about 250 g/L, and wherein the sample is contacted with (S,S)-EDDS (optionally the trisodium salt thereof) at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the concentration of (S,S)-EDDS (optionally the trisodium salt thereof is from about 2.5 mM to about 100 mM, preferably from about 5 mM to about 50 mM, or between about 10 mM to about 25 mM, more preferably from about 15 mM to about 20 mM.

In certain embodiments, the concentration of albumin in the albumin-comprising sample is from about 50 g/L to about 200 g/L, or from about 100 g/L to about 170 g/L, preferably from about 135 g/L to about 155 g/L, more preferably about 140 g/L to about 150 g/L, and wherein the sample is contacted with (S,S)-EDDS (optionally the trisodium salt thereof), at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the concentration of (S,S)-EDDS (optionally the trisodium salt thereof) is from about 2.5 mM to about 100 mM, preferably from about 5 mM to about 50 mM, or between about 10 mM to about 25 mM, more preferably from about 15 mM to about 20 mM.

In certain embodiments, the chelating agent is EDTA, or a salt thereof, and wherein the EDTA is contacted with the albumin-comprising sample at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the EDTA is contacted with the albumin-comprising sample at a concentration of from about 10 mM to about 20 mM, preferably about 15 mM.

In certain embodiments, the chelating agent is EDTA or a salt thereof, and wherein the EDTA concentration is less than 100 mM, preferably less than 50 mM, or less than 25 mM, or less than 15 mM, or less than 5 mM. Most preferably, the concentration of EDTA is less than 1 mM or less than 500 μM, or less than 250 μM or less than 100 μM or 50 μM or less

In certain embodiments, the concentration of EDTA to be used is calculated relative to the concentration of copper in the albumin-comprising sample. As such, in certain embodiments, the EDTA is contacted with the albumin-comprising sample at an amount that corresponds to approximately at least a 2-fold higher concentration of EDTA to copper, at least a 5-fold higher concentration of EDTA to copper, at least a 10-fold higher concentration of EDTA to copper, at least a 15-fold higher concentration of EDTA to copper, at least a 20-fold higher concentration of EDTA to copper, at least a 25-fold higher concentration of EDTA to copper or at least at least a 50-fold higher concentration of EDTA to copper, at least a 75-fold higher concentration of EDTA to copper, at least a 100-fold higher concentration of EDTA to copper, at least a 125-fold higher concentration of EDTA to copper, at least a 150-fold higher concentration of EDTA to copper, at least a 175-fold higher concentration of EDTA to copper, or at least a 200-fold higher concentration of EDTA to copper, or higher.

In certain embodiments, the concentration of albumin in the albumin-comprising sample is from about 40 g/L to about 250 g/L, and wherein sample is contacted with EDTA (or salt thereof) at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the concentration of EDTA (or salt thereof) is from about 2.5 mM to about 100 mM, preferably from about 5 mM to about 50 mM, or between about 10 mM to about 25 mM, more preferably from about 15 mM to about 20 mM.

In certain embodiments, the concentration of albumin in the albumin-comprising sample is from about 50 g/L to about 200 g/L, or from about 100 g/L to about 170 g/L, preferably from about 135 g/L to about 155 g/L, more preferably about 140 g/L to about 150 g/L, and wherein sample is contacted with EDTA (or salt thereof) at a concentration of from about 10 μM to about 100 mM, preferably from about 25 μM to about 15 mM, more preferably from about 50 μM to about 1 mM, most preferably from about 50 μM to about 500 μM, especially about 50 μM. In alternative embodiments, the concentration of EDTA (or salt thereof) is from about 2.5 mM to about 100 mM, preferably from about 5 mM to about 50 mM, or between about 10 mM to about 25 mM, more preferably from about 15 mM to about 20 mM.

Preferably, the chelating agent used, and amount thereof, is sufficient to deplete the metal cations, preferably cupric ions, from the albumin-comprising sample to no more than about 5 μg metal cation/g protein, no more than 2.5 μg metal cation/g protein, no more than 2 μg metal cation/g protein, no more than 1 μg metal cation/g protein, preferably no more than about 0.8 μg/g protein. For example, where the metal cation to be depleted is cupric ion, the chelator used and amount thereof preferably depletes the albumin-comprising fraction of copper such that the amount of copper remaining in the metal cation-depleted comprising fraction is no more than about 5 μg copper/g protein, no more than 2.5 μg copper/g protein, no more than about 2 μg copper/g protein, no more than 1 μg copper/g protein, preferably no more than about 0.8 μg copper/g protein, more preferably, no more than 0.5 μg copper/g protein.

In a second aspect, the chelating agent may be provided in the form of a chelating resin. As such, the invention provides a process for preparing a purified albumin composition from an albumin-comprising sample obtained from blood-derived plasma, the process comprising:
i) providing an albumin-comprising sample obtained from blood-derived plasma;
ii) contacting the albumin-comprising sample with a chelating resin for sequestering metal cations to obtain a metal cation-depleted albumin-comprising sample;
thereby preparing a purified albumin composition.

In a further embodiment of the second aspect, there is provided a process for preparing a purified albumin composition from an albumin-comprising sample obtained from blood-derived plasma, the process comprising:
i) providing an albumin-comprising sample obtained from blood-derived plasma;
ii) contacting the albumin-comprising sample with a chelating resin for sequestering metal cations under conditions that allow metal cations but not albumin to bind the resin,
iii) recovering a metal cation-depleted albumin-comprising fraction;
thereby preparing a purified albumin composition.

In preferred embodiments of the second aspect of the invention, the chelating resin is a cation exchange resin.

The cation exchange resin may be a strong cation exchanger (i.e., strongly acidic) or a weak cation exchanger (i.e., weakly acidic). Preferably, the cation exchange resin comprises acidic moieties including sulfonic acid, or carboxylic acid groups.

It will be appreciated that the resin comprises a pore size that excludes or prevents albumin from contacting the negatively charged groups on the cation-exchange resin (and therefore is such that the resin is not bound by albumin), particularly when the pH is below the isolelectric point of albumin (which is approximately pH 4.8).

Non-limiting examples of suitable chelating resins for use in accordance with the invention include: Dowex® chelating resin A-1, also known as Chelex 100, which is based on iminodiacetic acid in a styrene-divinylbenzene matrix. Other functional groups bound to chelating resins include aminophosphonic acids, thiourea, and 2-picolylamine.

In other examples, the cation resin may be AG® (or Dowex®) 50W cation exchange resin, having varying degrees of cross-linkage (e.g, AG® 50W-X2, AG® 50W-X4, AG® 50W-X8 and AG® 50W-X12, AG® 50W-X16 having 2%, 4%, 8% or 12% crosslinkage, respectively).

In certain preferred embodiments, the cation exchange resin may comprise sulfonic acid functional groups attached to a styrene divinylbenzene copolymer lattice.

In any embodiment of the first or second aspects of the invention, the contacting of the albumin-comprising sample with the ligand for binding to or for sequestering metal cations can be performed using a column-based chromatography method, or utilising a batch method.

It will be understood that the albumin-comprising sample of plasma used in any of the present methods may be any sample of plasma obtained from blood (or a blood derivative such as serum or plasma), and which contains albumin.

The term “blood derived plasma” refers to the straw-coloured/pale yellow component of blood obtained from one or more blood donor(s). Methods of obtaining plasma from a donor will be apparent to a skilled person and/or described herein. In embodiments blood derived plasma is obtained by removing red blood cells from donated blood. In alternate embodiments, the blood derived plasma is obtained by plasmapheresis. In some embodiments the blood derived plasma is obtained by removing red blood cells from donated blood and/or plasmapheresis.

The albumin-comprising sample may be any intermediate or derivative obtained from any plasma fractionation process. In certain embodiments, the albumin-comprising sample may be an intermediate obtained from a plasma salt-precipitation process. Alternatively, the albumin-comprising sample may be an intermediate or fraction obtained by alcohol fractionation of blood derived plasma.

In preferred embodiments of either the first or second aspects of the invention, the albumin-comprising sample is a Cohn Fraction or a Kistler-Nitschmann Fraction or similar, obtained from cold-ethanol fractionation of blood derived plasma. In embodiments the albumin-comprising sample is selected from the list consisting of Cryosupernatant, Cohn Supernatant I, Cohn Supernatant (I+)II+III, Filtrate A, Fraction IV filtrate, Fraction IV₁ filtrate, or Fraction IV₄ filtrate. In particularly preferred embodiments, the albumin-comprising sample is Cohn Fraction V or Kistler-Nitschmann Precipitate C, or a suspension, filtrate or concentrate thereof. An example of a suspension, filtrate or concentrate of Precipitate C or of Fraction V may be referred to herein as Filtrate D. In alternative embodiments, the albumin-comprising sample is a chromatographic purified fraction. Examples of such fractions are illustrated in FIG. 1.

In preferred embodiments, the pH of the albumin-comprising sample is between about 4.6 to about 4.8, particularly when the albumin-comprising sample is Cohn Fraction V or Kistler-Nitschmann Precipitate C.

Alternatively, the pH of the albumin-comprising sample may be in the neutral range, preferably between about 6.8 to about 7.2, more preferably between about 7.0 to about 7.2, particularly when the albumin-comprising sample comprises a filtrate of chromatographic purified fraction of Cohn Fraction V or Kistler-Nitschmann Precipitate C.

In any aspect of the invention, the process comprises resuspending a precipitate comprising an albumin-comprising sample of blood, prior to contacting the sample with the ligand for binding to or for sequestering metal cations. Optionally, the resuspending is in water for injection (WFI).

In any aspect, the metal cation-depleted albumin-comprising sample contains at least 10%, 20%, 30%, 40% or 50% less metal cations than the albumin-comprising sample obtained from blood prior to contacting with a ligand for binding to or for sequestering metal cations. In other words, the level of metal cations in the metal cation-depleted albumin-comprising sample is reduced by at least about 10%, 20%, 30%, 40% or 50% compared to the albumin-comprising sample obtained from blood prior to contacting with a ligand for binding to or for sequestering metal cations.

In particular embodiments of the first or second aspect of the invention, the methods may further comprises an optional step of acidification. The acidification may occur before or after the albumin-comprising sample is contacted with the ligand for binding to or for sequestering metal cations. When the ligand is a chelating agent, preferably the step of acidification is performed after the step of contacting the albumin-comprising sample with the chelating agent. When the ligand is a cation-exchange resin, preferably the step of acidification is performed prior to contacting the albumin-comprising sample with the cation-exchange resin.

Acidification is preferably performed by contacting the albumin-comprising sample (or the albumin-comprising sample depleted of metal cations) with an inorganic acid, optionally selected from the group consisting of: sulphuric acid (H₂SO₄), citric acid (C₆H₈O₇), hydrochloric acid (HCl), phosphoric acid (H₃PO₄), oxalic acid (C₂H₂O₄) and formic acid (CH₂O₂). In a preferred embodiment, acidification is performed using sulphuric acid or hydrochloric acid.

Preferably, and particularly in embodiments where the albumin-comprising sample is Cohn Fraction V or Kistler-Nitschmann Precipitate C, the pH of the sample may typically be in the range of between about 4.6 to about 5.0 prior to the addition of chelating agent. In such embodiments, an optional step of acidification may be performed after the addition of chelating agent, resulting in a reduction in the pH of the albumin-comprising sample to a pH of approximately 3.0 to 4.5, preferably to a pH of between about 3.5 to about 4.5, or between about 3.6 to 4.4, or between about 3.7 to about 4.3, or between about 3.8 to about 4.2, most preferably between about 3.9 to about 4.2. In a preferred embodiment, the step of acidification results in a reduction in the pH of the albumin-comprising sample to a pH of about 3.9, about 4.0, about 4.1 or about 4.2.

In preferred embodiments, the pH of the albumin-comprising sample that is depleted of metal cations is no lower than about 4.5, no lower than about 4.2 or no lower than about 4.0 following acidification.

In embodiments where the albumin-comprising sample is a filtrate of Cohn Fraction V or Kistler-Nitschmann Precipitate C, (such as Filtrate D), the pH of the sample may be in the neutral range prior to the addition of chelating agent. Accordingly, in such embodiments, prior to addition of the chelating agent, the process comprises an acidification step to adjust the pH to about 5.6 to about 6.0, more preferably to about 5.8 or about 5.9

In any aspect of the invention, the process further comprises subjecting the albumin-comprising sample that is depleted of metal cations (or the acidified albumin-comprising sample that is depleted of metal cations) to additional purification steps. In some embodiments, the process therefore comprises subjecting the albumin-comprising sample that is depleted of metal cations (or the acidified albumin-comprising sample that is depleted of metal cations) to a pre-filtration step (e.g., clarifying depth filtration), ultrafiltration (e.g., diafiltration and/or concentration) and combinations thereof.

In a particularly preferred embodiment of the first aspect of the invention, there is provided a process for preparing a purified albumin pharmaceutical composition from an albumin-comprising sample obtained from blood-derived plasma, the process comprising:
i) providing an albumin-comprising sample obtained from blood-derived plasma, preferably wherein the sample is Cohn Fraction V or Kistler-Nitschmann Precipitate C, or a suspension, filtrate or concentrate thereof;
ii) contacting the albumin-comprising sample with a biodegradable chelating agent to obtain a metal cation-depleted albumin-comprising sample, wherein the biodegradable chelating agent is preferably (S,S)-EDDS, or a salt thereof;
iii) optionally, adjusting the pH of the sample prior to or immediately after the contacting with the chelating agent;
iv) recovering the metal cation-depleted albumin-comprising sample,

thereby preparing a purified albumin composition. Preferably, the process further comprises a subsequent step of diafiltration to remove or substantially deplete any remaining chelating agent from the sample. Preferably, the (S,S)-EDDS or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the (S,S)-EDDS is added to a concentration of from about 25 μM to about 20 mM, preferably from about 50 μM to about 15 mM, more preferably from about 50 μM to about 500 μM, most preferably about 50 μM.

In a particularly preferred embodiment of the first aspect of the invention, the invention relates to a process comprising:
i) providing an albumin-comprising sample obtained from blood-derived plasma, preferably wherein the sample is Cohn Fraction V or Kistler-Nitschmann Precipitate C;
ii) contacting the albumin-comprising sample with a biodegradable chelating agent to obtain a metal cation-depleted albumin-comprising sample, wherein the biodegradable chelating agent is preferably (S,S)-EDDS, or a salt thereof;
iii) optionally, adjusting the pH of the sample after the contacting with the chelating agent, wherein preferably, the pH is adjusted to a pH of about 3.9 to about 4.3, more preferably, about 4.1 to 4.2;
iv) recovering the metal cation-depleted albumin-comprising sample,

thereby preparing a purified albumin composition. Preferably, the process further comprises a subsequent step of depth filtration, and pH adjustment to a pH of about 7.0 to about 7.4, preferably about 7.2 to about 7.3, preferably about 7.2, followed by diafiltration to remove or substantially deplete any remaining chelating agent from the sample. Preferably, the (S,S)-EDDS or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the (S,S)-EDDS is added to a concentration of from about 25 μM to about 20 mM, preferably from about 50 μM to about 15 mM, more preferably from about 50 μM to about 500 μM, most preferably about 50 μM.

Further still, in another preferred embodiment of the first aspect of the invention, the invention relates to a process comprising:
i) providing an albumin-comprising sample obtained from blood-derived plasma, preferably wherein the sample is a filtrate of Cohn Fraction V or Kistler-Nitschmann Precipitate C (eg Filtrate D);
ii) optionally, adjusting the pH of the sample wherein preferably, the pH is adjusted to a pH of about 5.6 to 6.0, more preferably about 5.8 or about 5.9;
iii) contacting the albumin-comprising sample with a biodegradable chelating agent to obtain a metal cation-depleted albumin-comprising sample, wherein the biodegradable chelating agent is preferably (S,S)-EDDS, or a salt thereof;
iv) recovering the metal cation-depleted albumin-comprising sample,

thereby preparing a purified albumin composition. Preferably, the process further comprises a subsequent step of diafiltration to remove or substantially deplete any remaining chelating agent from the sample. Preferably, the (S,S)-EDDS or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the (S,S)-EDDS is added to a concentration of from about 25 μM to about 20 mM, preferably from about 50 μM to about 15 mM, more preferably from about 50 μM to about 500 μM, most preferably about 50 μM.

In a further embodiment of the first aspect of the invention, there is provided a process for preparing a purified albumin pharmaceutical composition from an albumin-comprising sample obtained from blood-derived plasma, the process comprising:
i) providing an albumin-comprising sample obtained from blood-derived plasma, preferably wherein the sample is Cohn Fraction V or Kistler-Nitschmann Precipitate C, or a suspension, filtrate or concentrate thereof;
ii) contacting the albumin-comprising sample with a chelating agent to obtain a metal cation-depleted albumin-comprising sample, wherein the chelating agent is preferably EDTA, or a salt thereof;
iii) optionally, adjusting the pH of the sample prior to or immediately after the contacting with the chelating agent;
iv) recovering the metal cation-depleted albumin-comprising sample,

thereby preparing a purified albumin composition. Preferably, the process further comprises a subsequent step of diafiltration to remove or substantially deplete any remaining chelating agent from the sample. Preferably, the EDTA or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the EDTA is added to a concentration of from about 25 μM to about 20 mM, preferably from about 50 μM to about 15 mM, more preferably from about 50 μM to about 500 μM, most preferably about 50 μM.

In an embodiment of the first aspect of the invention, the invention relates to a process comprising:
i) providing an albumin-comprising sample obtained from blood-derived plasma, preferably wherein the sample is Cohn Fraction V or Kistler-Nitschmann Precipitate C;
ii) contacting the albumin-comprising sample with a chelating agent to obtain a metal cation-depleted albumin-comprising sample, wherein the chelating agent is preferably EDTA, or a salt thereof;
iii) optionally, adjusting the pH of the sample after the contacting with the chelating agent, wherein preferably, the pH is adjusted to a pH of about 3.9 to about 4.3, more preferably, about 4.1 to 4.2;
iv) recovering the metal cation-depleted albumin-comprising sample,

thereby preparing a purified albumin composition. Preferably, the process further comprises a subsequent step of depth filtration, and pH adjustment to a pH of about 7.0 to about 7.4, preferably about 7.2 to about 7.3, preferably about 7.2, followed by diafiltration to remove or substantially deplete any remaining chelating agent from the sample. Preferably, the EDTA or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the EDTA is added to a concentration of from about 25 μM to about 20 mM, preferably from about 50 μM to about 15 mM, more preferably from about 50 μM to about 500 μM, most preferably about 50 μM.

Further still, in another embodiment of the first aspect of the invention, the invention relates to a process comprising:
i) providing an albumin-comprising sample obtained from blood-derived plasma, preferably wherein the sample is a filtrate of Cohn Fraction V or Kistler-Nitschmann Precipitate C (eg Filtrate D);
ii) optionally, adjusting the pH of the sample wherein preferably, the pH is adjusted to a pH of about 5.6 to 6.0, more preferably about 5.8 or about 5.9;
iii) contacting the albumin-comprising sample with a chelating agent to obtain a metal cation-depleted albumin-comprising sample, wherein the chelating agent is preferably EDTA, or a salt thereof;
iv) recovering the metal cation-depleted albumin-comprising sample,

thereby preparing a purified albumin composition. Preferably, the process further comprises a subsequent step of diafiltration to remove or substantially deplete any remaining chelating agent from the sample. Preferably, the EDTA or salt thereof is added to a final concentration of at least about a 2-fold, at least about a 5-fold or at least about a 10-fold, at least about a 50-fold, at least about a 100-fold or at least about a 200-fold higher concentration than the concentration of copper in the sample; optionally wherein the EDTA is added to a concentration of from about 25 μM to about 20 mM, preferably from about 50 μM to about 15 mM, more preferably from about 50 μM to about 500 μM, most preferably about 50 μM.

The present invention also provides a purified albumin product comprising a concentration of copper of no more than about 2.0 μg/g protein, no more than about 1.5 μg/g protein, no more than about 1 μg/g protein, no more than about 0.8 μg/g protein, no more than about 0.5 μg/g protein or no more than about 0.2 μg/g protein.

The present invention also provides a purified albumin product obtained or obtainable by any process described herein. In preferred embodiments, the purified albumin product comprises a concentration of copper of no more than about 2.0 μg/g albumin, no more than about 1.5 μg/g albumin, no more than about 1 μg/g albumin, no more than about 0.8 μg/g albumin or no more than about 0.6 μg/g albumin, no more than about 0.5 μg/g albumin, or no more than about 0.2 μg/g albumin.

As used herein, reference to a purified albumin composition suitable for pharmaceutical use refers to a composition which meets the pharmacopoeia standards, such as Ph. Eur, or USP or as otherwise herein defined. Preferably, a purified albumin composition suitable for pharmaceutical use is a composition that meets the pharmacopoeia standards, such as Ph. Eur, or USP, and which is specifically formulated for administration to humans.

The purified albumin products described herein may be formulated into an hSA drug product or pharmaceutical composition and to contain appropriate amounts of albumin as the active ingredient according to pharmacopeia standards such as Ph. Eur. or USP. The following excipients may be included: sodium N-acetyl-tryptophan (stabiliser); sodium caprylate (stabiliser); sodium chloride (tonicity agent); water for injections (solvent). The protein content may be adjusted as required to manufacture 4%, 5%, 20% and 25% Human Albumin Solution (hSA).

In particular embodiments the final hSA formulations are selected from the group consisting of: i) Protein (4% w/v), sodium (140 mM) and caprylate (6.4 mM) for 4% w/v hSA; ii) Protein (5% w/v), sodium (140 mM) and caprylate (8 mM) for 5% w/v hSA; iii) Protein (20% w/v) and caprylate (32 mM) for 20% w/v hSA; iv) Protein (25% w/v) and caprylate (40 mM) for 25% w/v hSA.

In further embodiments the final hSA formulations may be selected from the group consisting of: i) Protein (4% w/v), 3.2 mM sodium N-acetyltryptophanate and 3.2 mM sodium caprylate for 4% w/v hSA; ii) Protein (5% w/v), 4 mM sodium N-acetyltryptophanate and 4 mM sodium caprylate for 5% w/v hSA; iii) Protein (20% w/v) 0.016 M sodium N-acetyltryptophanate and 0.016 M sodium caprylate for 20% w/v hSA; iv) Protein (25% w/v) 0.02 M sodium N-acetyltryptophanate and 0.02 M sodium caprylate for 25% w/v hSA.

The hSA formulations preferably do not comprise preservatives.

An hSA pharmaceutical composition (drug product) according to the present invention will preferably meet the appropriate Pharmacopoeia standard. For example following the test procedures as described for a human albumin solution in the European Pharmocopoeia version 10.6 the hSA preparation is sterile; pyrogen free; has endotoxin levels below 0.5 IU per mL for solutions less than 50 g/L, or less than 1.3 IU per mL for solutions from 50 g/L to 200 g/L, or less than 1.7 IU/mL for solutions greater than 200 g/L; an aluminium content of a maximum of 200 μg/L, a prekallikrein activator (PKA) maximum of 35 IU/mL; a haem content not greater than 0.15; a potassium maximum of 0.05 mmol per gram of protein; a sodium maximum of 160 mmol/L and 95% to 105% of the content of Na stated on the label; a maximum of 10% polymers and aggregates; not more than 5% of protein has a mobility different from the principal band by zone electrophoresis; a pH of 6.7 to 7.3 and a total protein not less than 9% and not more than 10% of the stated content.

In any aspect of the invention, the albumin-comprising sample is obtained from human blood.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Schematic flow diagram comparing various methods for the preparation of plasma protein fraction and human albumin solution from blood derived plasma at industrial scale (from Br J Anaesth, Volume 85, Issue 6, 1 Dec. 2000, Pages 887-895).

FIG. 2: Example of dark sediments found in preparations of albumin.

FIG. 3: Copper concentration (μg/gram protein) after addition of 50 mM chelating agent to Filtrate D.

FIG. 4: Copper concentration (μg/gram protein) after addition of 50 mM chelating agent to Filtrate D, followed by pH adjustment to 5.8-5.9.

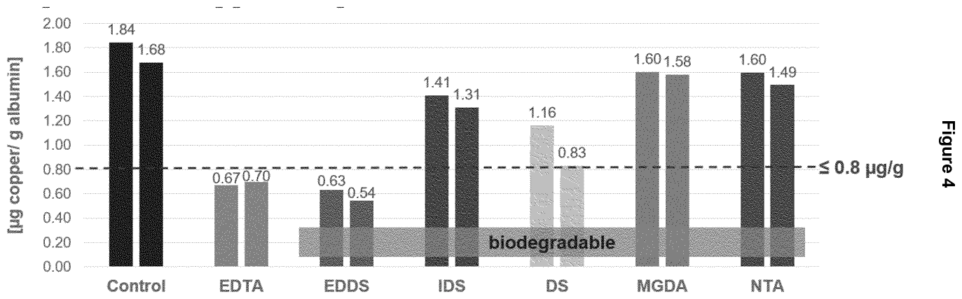

FIG. 5: Copper Concentration (mg/kg protein) following addition of various chelators (5-50 mM) to resuspended Precipitate C (Res C). Cont (control); (S,S)-EDDS at 15 mM, EDTA at 5 mM, IDS at 15 mM and 50 mM, NTA at 15 mM.

FIG. 6: Concentration of copper normalized to albumin concentration (μg copper/g albumin). Copper concentration in the paste resuspension, filtrate D and retentate (after UF/DF), is shown relative to (S,S)-EDDS concentration in the resuspension (0 mM to 15 mM).

FIG. 7: Concentration of copper normalized to albumin concentration (μg copper/g albumin). Copper concentrated in the paste resuspension, filtrate D and retentate (after UF/DF), is shown relative to (S,S)-EDDS concentration in the resuspension (0.05 mM to 1 mM).

FIG. 8: Copper reduction using acidification and Dowex cation exchange chromatography of Resuspended Precipitate C or Filtrate D.

FIG. 9: Accelerated stability study of albumin preparations obtained by various methods including acidification+cation exchange or addition of a chelator. Shown are albumin samples: not subjected to chelator treatment or cation exchange (control, at 8 month time point); subjected to acidification+cation exchange at 7 month time point (column and batch methods); subjected to treatment with EDTA (8 month time point); and subjected to treatment with (S,S)-EDDS (8 month time point).

FIG. 10: Accelerated stability study of albumin preparations obtained by addition of chelator. Shown are albumin samples: not subjected to chelator treatment (control; up to 12 months); or subjected to treatment with EDTA (5 mM pH 4.2 or 15 mM pH 4.7; up to 12 months); and subjected to treatment with (S,S)-EDDS (15 mM, pH 4.2; up to 12 months). Hashes=no dark sediment; no shading=very little dark sediment; dark shading=dark sediments clearly visible. Samples subjected to chelator treatment show only low levels of dark sediment formation 12 months after chelator treatment. In contrast, samples that are not treated with chelator show clear evidence of dark sediments within about 5 months.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Further aspects of the present invention and further embodiments of the aspects described in the preceding paragraphs will become apparent from the following description, given by way of example and with reference to the accompanying drawings.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

Human blood plasma has been industrially utilized for decades for the production of widely established and accepted plasma-protein products such as human albumin (hSA), immunoglobulin (IgG), clotting factor concentrates (clotting Factor VIII, clotting Factor IX, prothrombin complex etc.) and inhibitors (antithrombin, C1-inhibitor etc.). In the course of the development of such plasma-derived drugs, plasma fractionation methods have been established, leading to intermediate products enriched in certain protein fractions, which then serve as the starting composition for plasma-protein product/s. Typical processes are reviewed in e.g. Molecular Biology of Human Proteins (Schultze H. E., Heremans J. F.; Volume I: Nature and Metabolism of Extracellular Proteins 1966, Elsevier Publishing Company; p. 236-317).

hSA is routinely administered in clinical settings when there are existing or anticipated clinical problems or complications from reduced oncotic pressure, and/or as an adjunct to diuretic therapy. However, the formation of dark sediments and aggregates have been observed in different preparations of serum albumin. The present inventors have identified improved processes for the purification of albumin from serum, including methods which minimise the development of such sediments and aggregates.

Without being bound by theory, the inventors believe that the dark sediments are caused by metal cations bound to albumin and to other proteins such as ceruloplasmin, which are often present in albumin preparations. In particular, the present invention is based on the findings by the inventors that dark sediments are likely caused by the presence of copper (in the form of CuS, copper (II) sulfide) in albumin preparations, with proteolytic activity of contaminating proteases also contributing to aggregation. The present invention therefore seeks to address some of the limitations of prior art methods for the preparation of serum albumin products.

### General

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or groups of compositions of matter. Thus, as used herein, the singular forms “a”, “an” and “the” include plural aspects, and vice versa, unless the context clearly dictates otherwise. For example, reference to “a” includes a single as well as two or more; reference to “an” includes a single as well as two or more; reference to “the” includes a single as well as two or more and so forth.

Those skilled in the art will appreciate that the present invention is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

One skilled in the art will recognize many methods and materials similar or equivalent to those described herein, which could be used in the practice of the present invention. The present invention is in no way limited to the methods and materials described.

All of the patents and publications referred to herein are incorporated by reference in their entirety.

The present invention is not to be limited in scope by the specific examples described herein, which are intended for the purpose of exemplification only. Functionally-equivalent products, compositions and methods are clearly within the scope of the present invention.

Any example or embodiment of the present invention herein shall be taken to apply mutatis mutandis to any other example or embodiment of the invention unless specifically stated otherwise.

Unless specifically defined otherwise, all technical and scientific terms used herein shall be taken to have the same meaning as commonly understood by one of ordinary skill in the art.

The term “and/or”, e.g., “X and/or Y” shall be understood to mean either “X and Y” or “X or Y” and shall be taken to provide explicit support for both meanings or for either meaning.

By “about” or “approximately” in relation to a given numerical value for percentage, pH, amount or a period of time or other references, it is meant to include numerical values within 10% of the specified value.

### Starting Materials: Albumin-Comprising Fraction or Precipitate

It will be appreciated that the methods of the present invention find application in removing contaminating metal cations from any albumin-comprising sample of blood. Thus, the starting material for use in any of the methods described herein, can be any plasma-derived composition that comprises albumin.

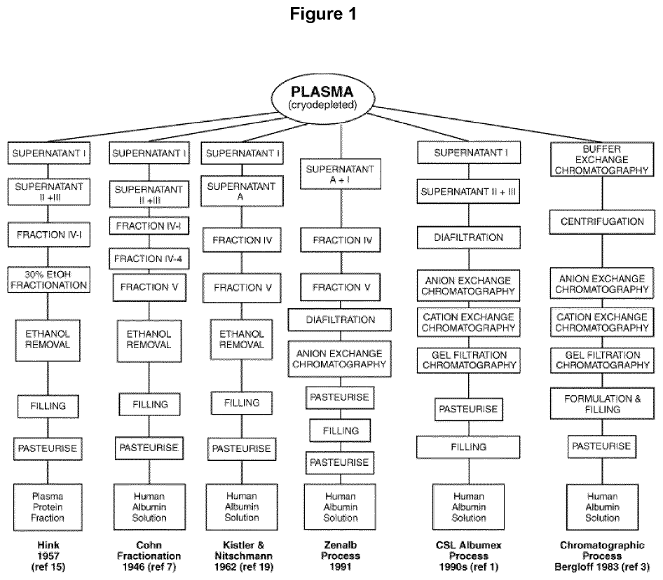

The skilled person will be familiar with various fractionation methods for obtaining albumin from blood. FIG. 1, herein, provides an overview of various fractionation methods for obtaining pharmaceutical compositions of albumin from blood derived plasma at an industrial scale. In order to obtain albumin from plasma (or serum), the plasma or serum is usually subjected to alcohol fractionation, which may be combined with other purification techniques like chromatography, adsorption or precipitation. However, other processes can also be used. For instance, the albumin-comprising precipitate can be precipitate V obtained according to the Cohn's methods such as the Method 6, Cohn et. al. J. Am; Chem. Soc., 68 (3), 459-475 (1946), the Method 9, Oncley et al. J. Am; Chem. Soc., 71, 541-550 (1946), or Precipitate-C of Nitschmann and Kistler Vox Sang. 7, 414-424 (1962); Helv. Chim. Acta 37, 866-873 (1954). Alternative precipitates comprising albumin include but are not limited to other albumin-comprising Oncley fractions, Cohn fractions, ammonium sulphate precipitates and other chromatographic methods as described by Schulze et al. in U.S. Pat. No. 3,301,842, Curling et al., (1977) Voc Saguinis, 33: 97-107; Tanaka et al., (1998) Braz. J. Med. Bio. Res, 31: 1383-1388, Raouifinia et al., (2016) Adv. Pharm. Bull, 6: 495-507; incorporated herein by reference.

“Normal plasma”, “hyperimmune plasma” (such as hyperimmune anti-D, tetanus or hepatitis B plasma) or any plasma equivalent thereto can be used as a starting material in the cold ethanol fractionation processes described herein.

The albumin-comprising composition or sample may be a cryosupernatant. The term ‘cryosupernatant’ (also called cryo-poor plasma, cryoprecipitate depleted plasma and similar) refers to plasma (derived from either whole blood donations or plasmapheresis) from which the cryoprecipitate has been removed. Cryoprecipitation is the first step in most plasma protein fractionation methods in use today, for the large-scale production of plasma protein therapeutics. The method generally involves pooling frozen plasma that is thawed under controlled conditions (e.g. at or below 6° C.) and the precipitate is then collected by either filtration or centrifugation. The supernatant fraction, known to those skilled in the art as a “cryosupernatant”, is generally retained for use. The resulting cryo-poor plasma has reduced levels of Factor VIII (FVIII), von Willebrand factor (VWF), Factor XIII (FXIII), fibronectin and fibrinogen. While the levels of FVIII are greatly reduced, levels of fibrinogen can be as much as 70% of original levels. Cryosupernatant provides a common feedstock used to manufacture a range of therapeutic proteins, including albumin.

The albumin-comprising composition or sample may be an ethanol precipitate. The supernatant of the 8% ethanol-precipitate (method of Cohn et al.; Schultze et al. (see above), p. 251), precipitate V (method of Oncley et al.; Schultze et al. (see above) p. 253) or precipitate C (method of Kistler and Nitschmann; Schultze et al. (see Schultze above), p. 253) are examples of a source of albumin compatible with industrial scale plasma fractionation. The source of albumin may also be any filtrate, concentrate or suspension of precipitate V or precipitate C (such as, for example, Filtrate D which is obtained from subjecting precipitate C to clarifying depth filtration).

Preferably, the total protein concentration of the starting material is between about 10-350 g/L, about 50-300 g/L, about 60 to about 250 g/L, about 75-150 g/L, most preferably about 50-100 g/L.

### Chelating Agents

In particular embodiments of the invention, the ligand for binding to or for sequestering metal cations in the albumin-comprising sample is a chelating agent. Preferably the chelating agent is used to deplete the albumin-comprising sample of copper ions, although it will be appreciated that the chelating agent can be used to deplete the protein fraction of other metal cations that may contribute to the formation of sediments in the final composition.

The skilled person will appreciate that any suitable chelating agent may be used in order to deplete the albumin-comprising sample of metal cations. In an embodiment the chelating agent binds to a divalent metal cation. In a preferred embodiment the chelating agent binds to copper (II) ions, wherein the copper is optionally present in the albumin solution as copper (II) sulfide.

Non-limiting examples of suitable chelators for use in the methods of the present invention include: ethylenediaminetetraacetic acid (EDTA), ethylene glycol-bis(3-aminoethyl ether)-N,N,N′,N′-tetraacetic acid (EGTA), ethylenediamine-N,N′-disuccinic acid (EDDS), iminodisuccinic acid (IDS), methylglycinediacetic acid (MGDA), triethylenetetramine (Trien), iminodiacetic acid (IDA), nitrilotriacetic acid (NTA), tripolyphosphate (TPP), diethylenetriaminepentaacetic acid (DTPA), sodium diethyldithiocarbamate (DDC), L-Glutamic acid N,N-diacetic acid, tetrasodium salt (GLDA) and penicillamine, of any salt thereof, including a calcium or sodium salt thereof.

In a particularly preferred embodiments, the chelating agent is selected from the group consisting of: ethylenediamine-N,N′-disuccinic acid (EDDS), iminodisuccinic acid (IDS), methylglycinediacetic acid (MGDA), and nitrilotriacetic acid (NTA), most preferably EDDS (especially (S,S)-EDDS), or a salt thereof, including a calcium or sodium salt thereof.

As used herein, ethylenediaminetetraacetic acid (EDTA, also known as 2,2′,2″,2″′-(Ethane-1,2-diyldinitrilo)tetraacetic acid, N,N′-Ethane-1,2-diylbis[N-(carboxymethyl)glycine], diaminoethane-tetraacetic acid and edetic acid; CAS no: 60-00-4), is a hexadendate ligand that binds to metal ions including Cu²⁺, Ca²⁺, Mg²⁺ and Fe³⁺. EDTA may be provided in free acid form, or as a salt, such as dehydrate disodium.

As used herein, ethylene glycol-bis(β-aminoethyl ether)-N,N,N′,N′-tetraacetic acid (EGTA, also known as egtazic acid, ethylene glycol-bis(2-aminoethylether)-N,N,N′,N′-tetraacetic acid, ethylene glycol tetraacetic acid and triethylene glycol diamine tetraacetic acid; CAS no: 67-42-5) is a hexadendate ligand that binds to metal ions including Cu²⁺, Ca²⁺, Mg²⁺ and Fe³⁺.

As used herein, ethylenediamine-N,N′-disuccinic acid (EDDS, CAS no: 20846-91-7) is an aminopolycarboxylic acid. It is a colourless solid that is used as chelating agent that may offer a biodegradable alternative to EDTA, which is currently used on a large scale in numerous applications. EDDS has two chiral centers, and as such three stereoisomers. These are the enantiomeric (R,R) and (S,S) isomers and the achiral meso (R,S) isomer. As a biodegradable replacement for EDTA, the (S,S) stereoisomer is preferred. Therefore in a preferred embodiment the chelating agent is (S,S)-EDDS. In a particularly preferred embodiment, the chelating agent is the trisodium salt of (S,S)-EDDS.

As used herein, IDS refers to iminodisuccinic acid, or a salt thereof, such as tetrasodium iminidisuccinate (also referred to as N-(1,2-dicarboxyethyl) aspartic acid; CAS no: 144538-83-0). Tetrasodium iminodisuccinate is a chelating agent, forming complexes of moderate stability which includes (as a pentadentate ligand) alkaline earth and polyvalent heavy metal ions with one molecule of water in an octahedral structure. Tetrasodium iminodisuccinate is classified as readily biodegradable according to OECD methods.

As used herein methylglycinediacetic acid (MGDA), usually provided in salt form: trisodium N-(1-carboxylatoethyl)iminodiacetate, methylglycinediacetic acid trisodium salt (MGDA-Na3) or trisodium α-DL-alanine diacetate (α-ADA), is the trisodium anion of N-(1-carboxyethyl)iminodiacetic acid and a tetradentate complexing agent. It forms stable 1:1 chelate complexes with cations having a charge number of at least +2, e.g. the “hard water forming” cations Ca²⁺ or Mg²⁺. Also known as α-ADA, this chelator is distinguished from the isomeric β-alaninediacetic acid by better biodegradability and therefore improved environmental compatibility.

As used herein triethylenetetramine (Trien or TETA, CAS no: 112-24-3), also called trientine (INN), is a chelating agent, typically when provided as a hydrochloride salt.

As used herein, iminodiacetic acid (IDA; CAS no: 142-73-4), is a dicarboxylic acid amine, of which the dianion is a tridentate ligand, forming metal complexes by forming two, fused, five membered chelate rings. The proton on the nitrogen atom can be replaced by a carbon atom of a polymer to create an ion-exchange resin, such as chelex 100.

As used herein, nitrilotriacetic acid (NTA, also known as N,N-Bis(carboxymethyl)glycine 2-[Bis(carboxymethyl)amino]acetic acid; Triglycine, CAS no: 139-13-9), is a colourless solid that is used as a chelating agent, which forms coordination compounds with metal ions (chelates) such as Ca²⁺, Co²⁺, Cu²⁺, and Fe³⁺ NTA is a tripodal tetradentate trianionic ligand.

As used herein, tripolyphosphate (TPP), also known as sodium triphosphate (STP), sodium tripolyphosphate (STPP), or Pentasodium triphosphate (CAS no: 7758-29-4) is an inorganic compound with formula Na₅P₃O₁₀. It is the sodium salt of the polyphosphate penta-anion, which is the conjugate base of triphosphoric acid. It is produced on a large scale as a component of many domestic and industrial products, especially detergents. It binds strongly to metal cations as both a bidentate and tridentate chelating agent.

As used herein, diethylenetriaminepentaacetic acid (DTPA), or pentetic acid (CAS no: 67-43-6) is an aminopolycarboxylic acid consisting of a diethylenetriamine backbone with five carboxymethyl groups. The molecule can be viewed as an expanded version of EDTA and is used similarly. It is a white solid with limited solubility in water.

As used herein, sodium diethyldithiocarbamate (DDC), (also known as Sodium diethylcarbamodithioate; CAS no: 148-18-5), coordinates to many “softer” metals via two sulfur atoms. Other more complicated bonding modes are known including binding as unidentate ligand and a bridging ligand using one or both sulfur atoms.

As used herein, L-Glutamic acid N,N-diacetic acid, tetrasodium salt (GLDA) is also known as tetrasodium glutamate diacetate (CAS no: 51981-21-6).

In any embodiment, the chelator may be provided in salt form, including a calcium, hydrochloride, or mono-, di-, tri-tetra-sodium salt thereof.

In certain embodiments, the chelator is combined with the albumin-comprising sample at a concentration of at least about a 2-fold, at least about a 5-fold, at least about a 10-fold, at least about a 25-fold, at least about a 50-fold, at least about a 100-fold or at least about a 200-fold higher concentration than the concentration of copper in the sample; optionally wherein the chelator is added to a concentration of between about 25 μM to about 20 mM, preferably between about 50 μM to about 15 mM, more preferably between about 50 μM to about 500 μM, most preferably about 50 μM.

In a preferred embodiment the biodegradable chelating agent is (S,S)-EDDS, and the (S,S)-EDDS is combined with the albumin-comprising sample at a concentration of at least about a 2-fold, or at least about a 5-fold, at least about a 10-fold, at least about a 25-fold, at least about a 50-fold, at least about a 100-fold or at least about a 200-fold higher concentration than the concentration of copper in the sample; optionally wherein the (S,S)-EDDS is added to a concentration of between about 25 μM to about 20 mM, preferably between about 50 μM to about 15 mM, more preferably between about 50 μM to about 500 μM, most preferably about 50 μM.

In certain embodiments, the chelator is combined with the albumin-comprising sample at a concentration of at least a 2-fold, at least a 5-fold, at least a 10-fold, at least a 25-fold, at least a 50-fold, at least a 100-fold or at least a 200-fold higher concentration than the concentration of copper in the sample; optionally wherein the chelator is added to a concentration of between 25 μM to 20 mM, preferably between 50 μM to 15 mM, more preferably between 50 μM to 500 μM, most preferably 50 μM.

In a preferred embodiment the biodegradable chelating agent is (S,S)-EDDS, and the (S,S)-EDDS is combined with the albumin-comprising sample at a concentration of at least at least a 2-fold, at least a 5-fold, at least a 10-fold, at least a 25-fold, at least a 50-fold, at least a 100-fold or at least a 200-fold higher concentration than the concentration of copper in the sample; optionally wherein the (S,S)-EDDS is added to a concentration of between 25 μM to 20 mM, preferably between 50 μM to 15 mM, more preferably between 50 μM to 500 μM, most preferably 50 μM.

### Resins for Chelating and Cation Exchange

Charged molecules such as proteins, peptides, and/or amino acids, as well as other solutes, may be separated from compositions by means of ion exchange. Ion exchange materials, such as ion exchange resins, typically comprise two types of ions: ions bound within or on the resin, and an oppositely charged counterion. When the resin is contacted with a composition, charged molecules in the composition may displace the counterions and bind to the resin. During the separation process, the charged molecules bound to the resin may be competitively and sequentially displaced or eluted from the resin in an order that is inversely related to the binding affinity of the charged molecules by raising the concentration of counterions in the sample. For more a more detailed discussion of ion exchange, see Moore, et al., “Chromatography of amino acids on sulfonated polystyrene resins,” 1958, Analytical Chemistry, 30:1185-1190.

In general, there are four major types of ion exchange resins or media. Strong cation exchange resins/media are strongly acidic, and generally contain fully ionized acidic groups such as sulfonic acid groups or the corresponding salts. These exchangers are negatively charged, and bind cations very strongly. The exchange capacity of strong acid or base resins is typically independent of the pH of the sample contacting the resin. Examples of strong cation exchangers include: sulfonic acid, Trisacryl, sulphopropyl, and the like.

Weak cation exchange resins/media contain weak acids, such as carboxylic acid groups or the corresponding salts (e.g., carboxymethyl (CM) cellulose, Chelex-100, etc.). Since the degree of dissociation of a weak acid resin is influenced by pH, resin capacity depends in part on solution pH. For example, a typical weak acid resin has very limited capacity below a pH of 6.0. A weak cation exchanger therefore operates across a narrow pH range (between about 6 and 7). Thus, by “weak cation exchange resin” or “weak cation exchange material” it is meant that the exchanger is a weak acid that will be negatively charged when the pH is above the pK a of the exchanger. Examples of weak cation exchangers include carboxymethyl (CM), phosphono and polyaspartic acid and the like.

Ion exchange resins may comprise a variety of charged groups. For example, cation exchange resin may comprise charged groups such as carboxymethyl (CM), sulfopropyl (SP), and/or methyl sulfonate (S). These charged groups may be attached to a variety of core materials including those that are agarose-based (e.g., SEPHAROSE CL-6B, SEPHAROSE FAST FLOW, and SEPHAROSE HIGH PERFORMANCE), cellulose-based (e.g., DEAE SEPHACEL), dextran-based (e.g., SEPHADEX), silica-based, and synthetic polymer based.

Although any of a variety of ion exchangers may be used in accordance with the methods described herein to bind soluble, charged proteins, peptides, amino acids, and/or other charged molecules, preferably the ion exchangers are strong cation exchangers (i.e., strongly acidic resins). In one embodiment, the cation exchange resin used herein preferably comprises a sulfonic cation exchange resin, for example comprising sulfonic acid (—SO₃H) bound to matrix styrene divinylbenzene. In other embodiments, the cation exchange resin may comprise an iminodiacetatic acid chelating cation exchange resin.

Although discussed herein primarily in terms of free-flowing resin, it is to be understood that the cation exchange material used in the methods described herein (ie, the ligand for binding to or for sequestering metal cations) may be in various formats. For example, the cation exchanger may be coated on beads, may be in the form of a membrane ion exchange resin, may be coated onto the interior surface or a portion of the interior surface of the container in which the separation is being performed, and/or may be coated on an object, such as a magnetic stir bar or rod, present in the container in which the separation is being performed. In other embodiments, the cation exchange material may be in the form of a strong acid cation exchange resin (H-form), or chelating ion exchange resin (amino phosphate) in a polyethylene matrix. Examples of such matrices include 3M® Metal Ion Purifiers filter cartridges and discs. Still further embodiments contemplate the use of an iminodiacetate functionalised poly[styrenedivinylbenzenel) resin, for example, Empore® (3M®) extraction discs. Other suitable formats for the ion exchangers will be apparent to those skilled in the art.

Typically, the cation exchange resin comprises a pore size that excludes albumin from contacting the negatively charged cation-exchange groups/ligands of the resin (such that preferably, the resin does not bind the albumin present in the albumin-comprising solution, particularly when the pH is below the isolelectric point of albumin (which is approximately pH 4.8). For example, preferably the cation exchange resin is a fine mesh resin for use in fine chemical and pharmaceutical column preparations.

In any embodiment, the mesh size of the cation exchange resin is in the range of between 50-400, preferably 50-200 or 50-100 (i.e., μm diameter equivalent of between 300 and 38 μm, preferably between 300 and 75 μm or most preferably between 300 and 150 μm, based on 1 μm=0.001 msm).

Because the aqueous dispersions described herein (e.g., comprising a composition comprising albumin and other proteins, ion exchange resin, and optionally a buffer or other pH adjusting agent) may be agitated to better facilitate separation of albumin and proteins, in one embodiment, the resin used herein is capable of free-flowing movement relative to the rest of the dispersion. For example, in this embodiment, the resin particles are preferably capable of independent movement relative both to other resin particles and relative to the other dispersion components. The aqueous dispersions comprising the free-flowing resin may therefore be amenable to agitation, in contrast to the relatively immobile state of resin that is packed in a column.

In certain embodiments of the invention, the ligand for binding to or for sequestering metal cations may comprise a cation exchange resin. Particularly preferred examples of suitable resins include cation exchange resins comprising a sulfonic cation exchange resin, for example comprising sulfonic acid (—SO₃H) bound to matrix styrene divinylbenzene. Commercially available examples of such resins are known to the skilled person and include the AG® or Dowex® fine mesh spherical strong acid cation exchange resins, especially the 50W series, and having varying degrees of resin cross-linking. In certain examples, the resin may be 50W X2, 50W X4, 50W X8, 50W X12, or 50W X16, having 2%, 4%, 8%, 12% or 16%, of divinylbenzene in the resin co-polymer, respectively.

Other examples of commercially available chelating resins that may be suitable for use in accordance with the present invention, include resins with an iminodiacetate functional group, such as Amberlite IRC 748, Lewatit TP 207, Lewatit TP 208, Purolite S 930, Lewatit MonoPlus TP 207, iminodiacetic acid fiber Ionex IDA-Na, and Dowex M 4195.

Other suitable resins will be apparent to those skilled in the art, and are described, for example, in Edebali and Pehlivan (2016) Powder Technology, 301: 520-525, incorporated herein by reference.

### Acidification

The methods of the invention may also comprise a step of acidification (a pH shift) to further improve the purity of the albumin-comprising sample and to minimise the impact of contaminating proteases.

In accordance with the first aspect of the invention, the step of acidification may be prior to or after the contacting of the albumin-comprising sample with the chelating agent. Optionally, wherein the albumin-comprising sample is Cohn Fraction V or Kistler-Nitschmann Precipitate C, the chelator is added to the sample first, followed by the step of acidification. Alternatively, when a filtrate of Precipitate C or Fraction V is used (e.g., Filtrate D), the step of acidification may be performed first, followed by addition of the chelating agent.

Preferably, the step of acidification comprises contacting the albumin-comprising sample (or the albumin-comprising sample depleted of metal cations) with an inorganic acid, optionally selected from the group consisting of: sulphuric acid (H₂SO₄), citric acid (C₆H₈O₇), hydrochloric acid (HCl), phosphoric acid (H₃PO₄), oxalic acid (C₂H₂O₄) and formic acid (CH₂O₂). In a preferred embodiment, acidification is performed using sulphuric acid or hydrochloric acid.

Preferably, when the acidification step occurs after the addition of the chelating agent, the step of acidification results in a reduction in the pH of the albumin-comprising sample to a pH of approximately 3.0 to 4.8, preferably to a pH of between about 4.0 to about 4.8, or between about 4.0 to 4.4, most preferably between about 3.9 to about 4.2.

Most preferably, when the acidification step occurs after the addition of the chelating agent, the step of acidification results in a reduction in the pH of the albumin-comprising sample to a pH of about 3.8, about 3.9, about 4.0, about 4.1, about 4.2, about 4.3, about 4.4, about 4.5, about 4.6, about 4.7 or about 3.8, especially to a pH of about 3.9, about 4.0, about 4.1 or about 4.2.

Alternatively, when the acidification step occurs prior to the addition of the chelating agent, (for example, when the albumin containing sample is a filtrate of Cohn Fraction V or Kistler-Nitschmann Precipitate C, which may have a pH in the range of about 7.0 to 7.5), the step of acidification results in a reduction in the pH of the albumin-comprising sample to a pH of approximately 5.6 to 6.0, preferably to a pH of between about 5.8 to about 5.9.

According to any aspect or embodiment of the invention, acidification optionally results in a reduction of the pH of the albumin-comprising sample to a pH of between about 4.6 to about 4.8.

Preferably, the inorganic acid is added to the albumin-comprising sample at a concentration of between about 0.01 M to about 5.0 M (molar), of between about 0.1 M to about 2.5 M, of between about 0.2 M to about 2.0 M, of between about 0.2 M to about 1.0 M, more preferably at a concentration of between about 0.25 M to about 0.5 M.

In particularly preferred embodiments, the inorganic acid is sulphuric acid or hydrochloric acid, and is added to the albumin-comprising sample at a concentration of between about 0.01 M to about 5.0 M (molar), of between about 0.1 M to about 2.5 M, of between about 0.2 M to about 2.0 M, of between about 0.2 M to about 1.0 M, more preferably at a concentration of between about 0.25 M to about 0.5 M.

It will be appreciated that prior to formulation of an hSA pharmaceutical composition, any chelating agent present in the albumin-comprising fraction should preferably be removed. Accordingly, in preferred embodiments, the present invention provides for various addition filtration methods following the step of recovering a metal cation-depleted albumin-comprising fraction according to any aspect of the invention.

In preferred embodiments, the subsequent steps of filtration may include a diafiltration step to remove any chelating agent, optionally including a concentration step prior to and/or following diafiltration. The skilled person will be familiar with suitable diafiltration methods for removal of undesirable components of a protein product. For example, the skilled person will be able to determine the number of rounds of diafiltration required (e.g, volumes of diafiltration) to remove the chelating agent. A diafiltration step may also be useful for removal of other contaminants including aluminium, other low molecular salts and ethanol.

Typically, the metal cation-depleted albumin product is subjected to further processing to prepare an hSA composition suitable for administration to a subject. For example, the metal-cation depleted albumin sample may be subjected to concentration, adjustment of sodium content (e.g, through addition of sodium chloride to a level acceptable for pharmacopoeial standards). Water for injection (WFI) may also be added to adjust the total protein concentration for final pharmaceutical composition (e.g., the protein content may be adjusted as required to manufacture 4%, 5%, 20% and 25% Human Albumin Solution (hSA).

The following excipients may also be included: sodium N-acetyl-tryptophan (stabiliser); sodium caprylate (stabiliser); sodium chloride (tonicity agent); water for injections (solvent).

The final albumin product is preferably also sterile filtered prior to dispensing and pasteurisation.

In particular embodiments the final hSA formulations are selected from the group consisting of: i) Protein (4% w/v), sodium (140 mM) and caprylate (6.4 mM) for 4% w/v hSA; ii) Protein (5% w/v), sodium (140 mM) and caprylate (8 mM) for 5% w/v hSA; iii) Protein (20% w/v) and caprylate (32 mM) for 20% w/v hSA; iv) Protein (25% w/v) and caprylate (40 mM) for 25% w/v hSA.

In further embodiments the final hSA formulations may be selected from the group consisting of: i) Protein (4% w/v), 3.2 mM sodium N-acetyltryptophanate and 3.2 mM sodium caprylate for 4% w/v hSA; ii) Protein (5% w/v), 4 mM sodium N-acetyltryptophanate and 4 mM sodium caprylate for 5% w/v hSA; iii) Protein (20% w/v) 0.016 M sodium N-acetyltryptophanate and 0.016 M sodium caprylate for 20% w/v hSA; iv) Protein (25% w/v) 0.02 M sodium N-acetyltryptophanate and 0.02 M sodium caprylate for 25% w/v hSA. The hSA formulations preferably do not comprise preservatives.

An hSA pharmaceutical composition according to the present invention will preferably meet the appropriate Pharmacopoeia standard. For example following the test procedures as described for a human albumin solution in the European Pharmocopoeia version 10.6 the hSA preparation is sterile; pyrogen free; has endotoxin levels below 0.5 IU per mL for solutions less than 50 g/L, or less than 1.3 IU per mL for solutions from 50 g/L to 200 g/L, or less than 1.7 IU/mL for solutions greater than 200 g/L; an aluminium content of a maximum of 200 μg/L, a prekallikrein activator (PKA) maximum of 35 IU/mL; a haem content not greater than 0.15; a potassium maximum of 0.05 mmol per gram of protein; a sodium maximum of 160 mmol/L and 95% to 105% of the content of Na stated on the label; a maximum of 10% polymers and aggregates; not more than 5% of protein has a mobility different from the principal band by zone electrophoresis; a pH of 6.7 to 7.3 and a total protein not less than 9% and not more than 10% of the stated content.

It will further be appreciated that the temperature at which the methods of the present invention are performed will be a temperature which maintains the integrity of the albumin product. Preferably, the methods are performed at a temperature of between 0° C. to about 20° C., preferably between 0° C. to about 10° C., more preferably between 2° C. to about 8° C., most preferably between 2° C. to about 4° C.

### EXAMPLES

### Example 1: Characterisation of Dark Sediment in Albumin Products

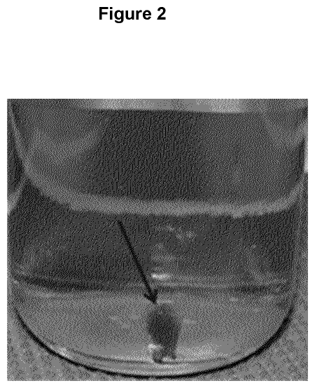

Dark sediment in Albumin is a known phenomenon; an example is shown in FIG. 2. The aim of this study was to investigate potential mechanisms leading to sediment formation, by characterization of:
(i) dark sediment isolated from Albumin and
(ii) Albumin 25% whole solution at end of shelf life with and without sediment as well as samples of fresh Albumin solutions.

Dark sediment found in certain albumin preparations, composed of hexagonal particles, was analyzed by scanning electron microscopy (SEM) combined with energy dispersive X-ray spectroscopy (EDX). The EDX analysis showed that the particles consist of copper and sulfur in an atomic proportion of approximately 1:1. It was hypothesised that CuS (copper sulfide) was formed over time of storage at 30° C.

Copper is present in the blood plasma and is not completely removed by the purification processes. A significant portion of the copper found in blood plasma is bound to the plasma proteins Ceruloplasmin and Albumin and it was therefore concluded that protein-bound copper was a likely contributor to dark sediment found in albumin preparations.

### Example 2: Use of a Chelating Step to Reduce Copper Levels in Albumin Preparations

An albumin-containing fraction was obtained from blood plasma according to the Kistler Nitschmann methods. Briefly:

Frozen plasma was thawed prior to adjusting the pH of the plasma to pH 5.7 to 6.0 with acetate acetic acid buffer (pH 4) in a stainless steel tank. Ethanol 96% was added continuously to a concentration of 19% (V/V) while stirring and cooling to a temperature of −5.5° C.±1.0° C. The pH was then adjusted to 5.70 to 5.90. The suspended precipitate A was removed by filtration after addition of perlite (filter aid) on polypropylene sheets. The filtrate of Precipitate A was collected in another stainless steel tank for further processing to albumin. The temperature was reduced to 7±0.5C, and simultaneously 96% ethanol was added to a final concentration of 40% (V/V). The pH of this solution was adjusted, if necessary, to 5.95 to 6.00. Fraction IV precipitates and was then, by the use of filter aids (perlite and diatomaceous earth), filtered through a filter matrix support in order to remove the precipitate.

The pH value of filtrate IV was adjusted by 1.1 M acetic acid to 4.8±0.1 at a constant ethanol concentration of 40% and under cooling to a temperature of 7±1° C. Precipitate C precipitates and was then separated from the ethanol solution by filtration with diatomaceous earth.

Precipitate C contains almost exclusively albumin and filter aids. For further processing Precipitate C from one or more fractionation lots was first resuspended in water for injection (1 kg paste+1.7 kg water for injection) and then filtered at pH 4.7±0.1 through asbestos free depth filters to obtain Filtrate D. The pH of Filtrate D was adjusted to 7.2±0.1 by means of 1 M NaOH.

Several chelators were selected for an initial series of experiments to determine if addition of chelating agent to an albumin-comprising plasma derivative, would be useful for depleting copper ions.

The chelators EDTA, EDDS (the (S,S) isomer thereof), EGTA, IDS, DS, MGDA and NTA were initially tested, when added at a concentration of 50 mM. Some of these complexing agents are already widely used in different areas like water softening or in washing and cleaning agents as a substitute for EDTA, e.g. MGDA, DTPA, and (S,S)-EDDS.

Two different approaches were followed to characterize the properties of the respective chelating agents. During the initial screen, individual chelators were added to Filtrate D. This was carried out with and without subsequent pH adjustment to pH 5.8-5.9.

Samples were taken at each intermediate stage and analyzed for the parameters of interest, namely copper, proteolytic activity and aggregate formation

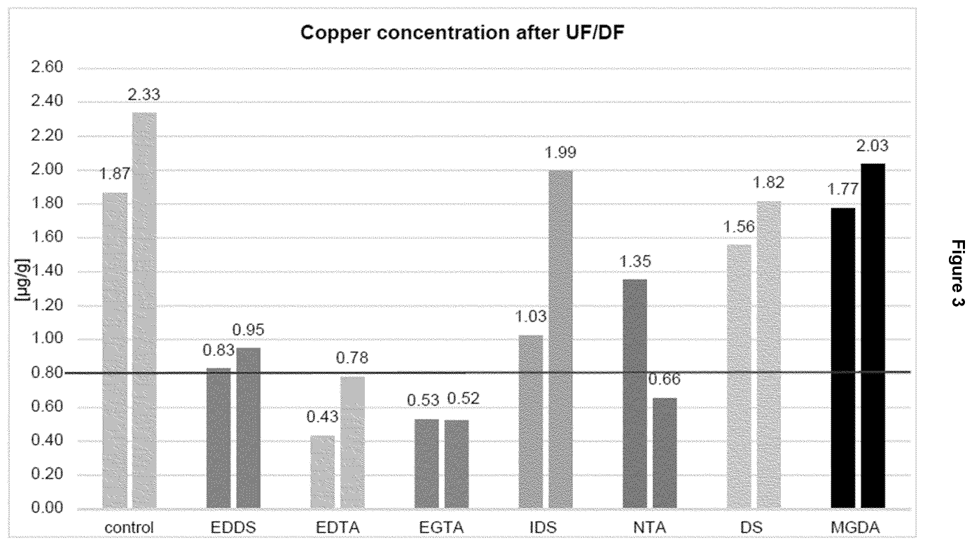

The results of the initial experiment shown in FIG. 3, demonstrate that addition of any of the chelators resulted in depletion of copper levels in the Filtrate D product. In particular, (S,S)-EDDS, EDTA, EGTA, and NTA reduced copper concentrations significantly, with (S,S), EDDS, EDTA and EGTA reducing copper levels close to or below the target of 0.8 μg copper/g protein.

Proteolytic activity after addition of chelators was assessed. The values were in the expected range for the ultrafiltrate process step (data not shown). The results from the initial screen indicates that addition of chelators caused a shift in pH. Consequently, it was decided to repeat the experiments with an adjustment of the pH of Filtrate D after addition of the chelators, to 5.8-5.9. pH was adjusted with sulphuric acid. The results are shown in FIG. 3 and indicate that several chelators retained their ability to deplete copper ions at this pH.

### Example 3: A Chelating Step is Required to Remove Copper Ions from Albumin Preparations

To demonstrate that chelation, rather than simply a pH change, is required to remove copper ions from albumin preparations, a further series of experiments were conducted.

Briefly, an albumin-containing sample was obtained according to the method described in Example 2 to obtain Filtrate D. Aliquots of Filtrate D were taken, and the pH adjusted to between 4.0 and 7.2. Samples of the aliquots were then subjected to filtration through a 10 kDa membrane (Amicon) and centrifugation at 2000 rpm for 110 minutes.

To exclude the possibility of an interaction of copper with the membrane, a positive control was also performed, using a solution of copper sulfate pentahydrate containing 0.5 mg/L of copper, which was also applied to a 10 kDa membrane and centrifuged at 2000 rpm.

The retentate and permeate following centrifugation were analysed to assess total protein concentration (g/L) and copper concentration (mg/kg).

The results demonstrate that when an albumin-comprising sample (in the form of Filtrate D is subjected to a range of different pH conditions (specifically in the range of pH 4.0 to 7.2), and then subjected to filtration across a 10 kDa membrane, copper ions are not detected in the permeate. The copper ions are found to remain in the retentate (bound to protein in the Filtrate). The results indicate that change in pH alone is not suitable for depleting copper present in albumin preparations, and that use of a chelator, as in Example 2, is required to deplete copper ions.

### Example 4: Use of Chelators in Scale-Up Manufacturing of Albumin Product

In a second series of experiments, the chelators were added to resuspended Precipitate C or Precipitate V, at commercial scale (ie in a large tank). The pH was adjusted to about 4.0-4.8 with 1 M HCl as needed.

The chelating agents were added at a concentration of 15 mM (5 mM for EDTA), because the results of Example 2 indicated that it is likely that a reduced amount of the chelating agent is required at a lower pH value.

The subsequent experiments were performed with the most promising candidates (S,S)-EDDS, IDS and NTA including EDTA as positive control.

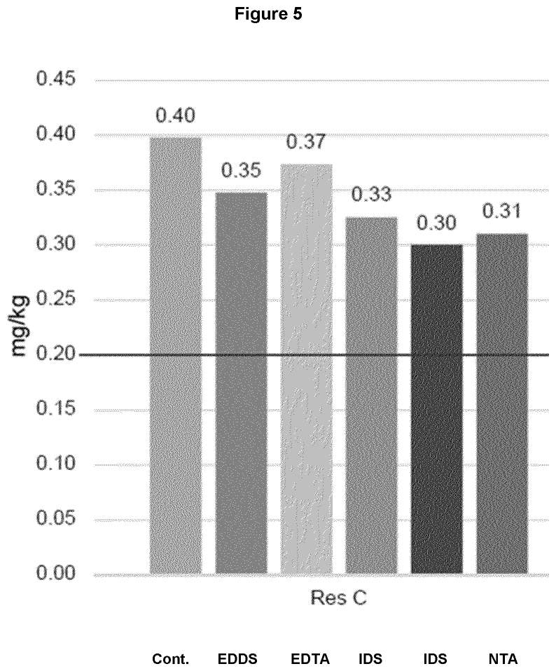

Comparable to the Filtrate D screening experiment, (S,S)-EDDS was determined to be the most promising biodegradable chelator, when added to the tank at the precipitate resuspension step with pH adjustment to 4.2 (FIG. 5).

Among the key findings of the experiments outlined in Examples 2 and 3 were that EDDS exhibited excellent copper depletion properties over the whole pH range investigated. This result is promising in terms of a certain flexibility with regards to the pH value, meaning that EDDS is not only effective at the lowest pH measured, but also at higher pH values up to 4.8.

### Example 5: Use of Alternative Concentrations of Chelator in Preparation of Albumin Product

A similar series of experiments to those conducted in Example 4 were performed, using the biodegradable chelator (S,S)-EDDS at a concentration of between 1 mM and 15 mM.

Briefly, (S,S)-EDDS (as a solution of Na₃EDDS) was added to resuspended Precipitate C (obtained from a KN fractionation process) and the pH was adjusted to about 4.6-4.8 with 1 M HCl as needed. The mixture was then processed through depth filtration and ultrafiltration steps to obtain Filtrate D.

The pH of Filtrate D was readjusted to pH 7.2-7.3 prior to ultrafiltration and then concentrated to 135±5 g protein/kg, and the subjected to diafiltration.

In order to understand the effect of different concentrations of EDDS in copper depletion, the concentration of this metal was measured throughout the process.

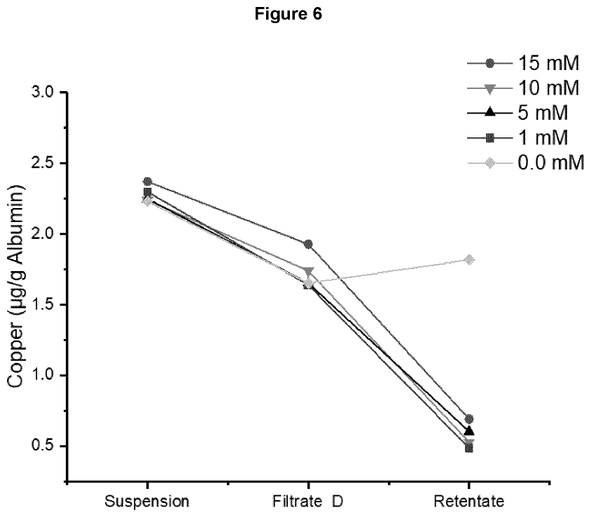

As shown in FIG. 6 constant depletion of copper was observed throughout the purification process. This confirmed the observations made in Example 4.

As shown in the below table, the extent of copper depletion ranged from 71% (using 15 mM (S,S)-EDDS) to 79% (using 1 mM (S,S)-EDDS).

| | EDDS | Copper (μg copper/g albumin) | | | % copper |
|---|---|---|---|---|---|
| Exp no. | (mM) | Suspension | Filtrate D | Retentate | depletion |
| 1 | 15 | 2.4 | 1.9 | 0.7 | 71 |
| 2 | 10 | 2.2 | 1.7 | 0.5 | 76 |
| 3 | 5 | 2.2 | 1.7 | 0.6 | 73 |
| 4 | 1 | 2.3 | 1.6 | 0.5 | 79 |
| 5 | 0 | 2.2 | 1.7 | 1.8 | 18 |
| (neg | | | | | |
| control) | | | | | |

The concentration of (S,S)-EDDS did not impact on the proteolytic activity in the end product, with all results being equal to or lower than 3 nkat/L.

There was no impact on the molecular size distribution of albumin (e.g. in the monomer and aggregate content) at the different concentrations of (S,S)-EDDS throughout the process.

Overall, the results demonstrate that when using lower concentrations of (S,S)-EDDS, from 15 mM to 1 mM, there is still a significant decrease in the level of copper in solution, including in resuspended Filtrate C, and filtrates thereof. A 71 to 79% decrease in the concentration of copper (normalized to the albumin concentration) was achieved in the studied range when (S,S)-EDDS was present, compared to an 18% decrease in the absence of chelator.

This study demonstrates that a 15-fold decrease in the concentration of the chelator added to the suspension achieves similar results regarding copper depletion, compared to the results discussed in Example 4.

In a similar series of experiments, the concentration of (S,S)-EDDS was kept constant at 15 mM, while varying the pH and ethanol concentration of the resuspension. No significant change in the results was observed.

### Example 6: Further Study of Alternative Concentrations of Chelator in Preparation of Albumin Product

A similar series of experiments to those conducted in Examples 4 and 5 were performed, using the biodegradable chelator (S,S)-EDDS at a concentration of between 0.05 mM and 1 mM.

Briefly, (S,S)-EDDS (as a solution of Na₃EDDS) was added to resuspended Precipitate C (obtained from a KN fractionation process) and the pH was adjusted to about 4.6-4.8 with 1 M HCl as needed. The mixture was then processed through depth filtration and ultrafiltration steps to obtain Filtrate D.

The pH of Filtrate D was readjusted to pH 7.2-7.3 prior to ultrafiltration and then concentrated to 135±5 g protein/kg, and the subjected to diafiltration.

In order to understand the effect of different concentrations of (S,S)-EDDS in copper depletion, the concentration of this metal was measured throughout the process.

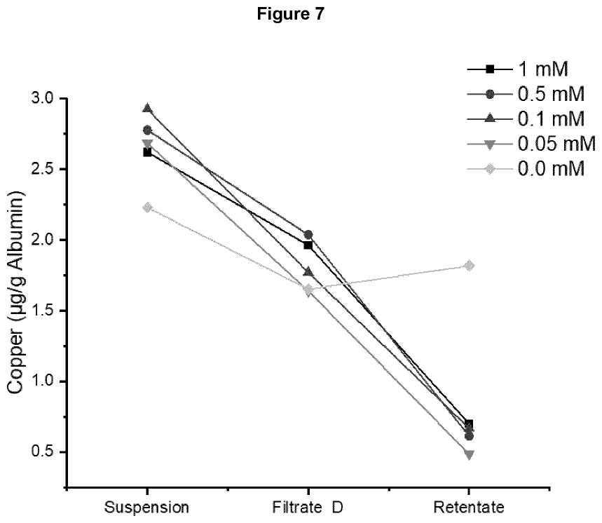

As shown in FIG. 7 constant depletion of copper was observed throughout the purification process. This confirmed the observations made in Examples 4 and 5.

As shown in the below table, the extent of copper depletion ranged from 73% (using 1 mM (S,S)-EDDS) to 82% (using 0.05 mM (S,S)-EDDS)

| | EDDS | Copper (μg copper/g albumin) | | | % copper |
|---|---|---|---|---|---|
| Exp no. | (mM) | Suspension | Filtrate D | Retentate | depletion |
| 1 | 1 | 2.6 | 2.0 | 0.7 | 73 |
| 2 | 0.5 | 2.8 | 2.0 | 0.6 | 78 |
| 3 | 0.1 | 2.9 | 1.8 | 0.7 | 77 |
| 4 | 0.05 | 2.7 | 1.6 | 0.5 | 82 |
| 5 | 0 | 2.2 | 1.7 | 1.8 | 18 |
| (neg | | | | | |
| control) | | | | | |

The concentration of (S,S)-EDDS did not impact on the proteolytic activity in the end product, with all results being equal to or lower than 3 nkat/L.

There was no impact on the molecular size distribution of albumin (e.g. in the monomer and aggregate content) at the different concentrations of (S,S)-EDDS throughout the process.

Overall, the results demonstrate that when using lower concentrations of (S,S)-EDDS, from 0.05 mM to 1 mM, there is still a significant decrease in the level of copper in solution, including in resuspended Filtrate C, and filtrates thereof. A 73% to 82% decrease in the concentration of copper (normalized to the albumin concentration) was achieved in the studied range when (S,S)-EDDS was present, compared to an 18% decrease in the absence of chelator.

Initial experiments using 15 mM of the chelator (Example 4) achieved a depletion of copper in the range of 70-80%, with further experiments using (S,S)-EDDS between 1 and 15 mM resulting in values in the same range (Example 5). Therefore, the results obtained in this study indicate that the chelator is equally effective in removing copper with 15 or 0.05 mM—a 300-fold decrease in the quantity added to the suspension. A possible explanation is that the initial concentration of copper in the paste suspension is approximately 0.4 mg/kg, which corresponds to a concentration of copper of 6 μM, meaning that 50 μM of the chelator in the suspension is still ˜10-fold excess compared to copper.

### Example 7: Use of Cation-Exchange Chromatography to Reduce Copper Levels in Albumin Preparations

In this example, the inventors studied whether cation exchange chromatography of Resuspended Precipitate C to cation-exchange chromatography would also deplete the albumin preparation of copper.

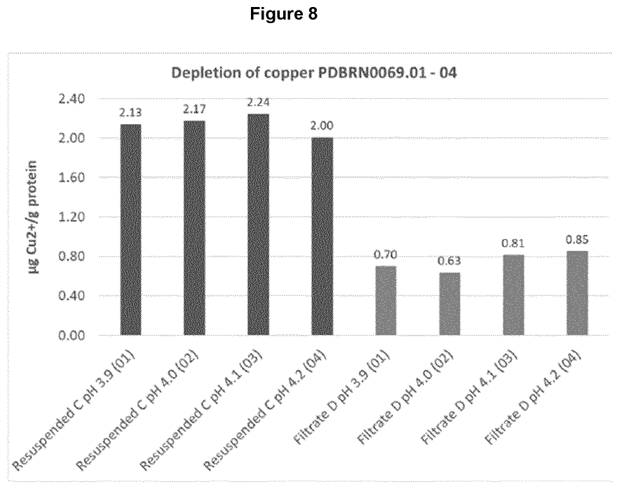

The study was conducted using a Dowex® cation exchange resin column packed to a bed height of 21.5 cm with a mixture of 50% Dowex mesh 200-400 resin & 50% Dowex® mesh 100-200 resin (Dowex® 50WX2) The column was operated at a constant residence time of 10 min during loading and post load wash for all pH set points. The results of this study are presented in FIG. 8. A clear trend was observed towards a reduced copper depletion in dependence of pH set point. The highest depletion was achieved at a pH 3.9, whereas the least was observed at a pH of 4.2 (slightly above target of ≤0.8 μg copper/g albumin).

### Example 8: Stability Study to Assess Sediment Formation

Various preparations of albumin were assessed over a period of several months to determine the impact of a chelator, or cation exchange process, to delay sediment formation.

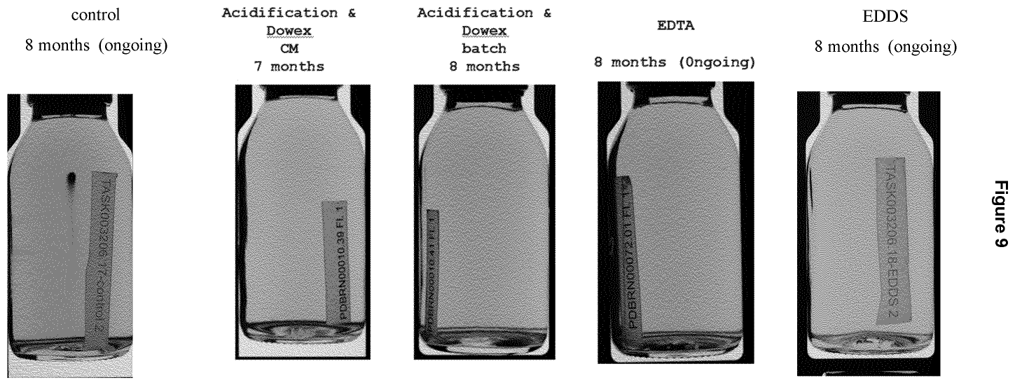

Preliminary results are shown in FIG. 9 and indicate that at least 8 months after contacting albumin-comprising samples with cation exchange resins (in batch or column chromatography), or with the chelators EDTA or (S,S)-EDDS, there is no evidence of the formation of sediment, compared to untreated control.

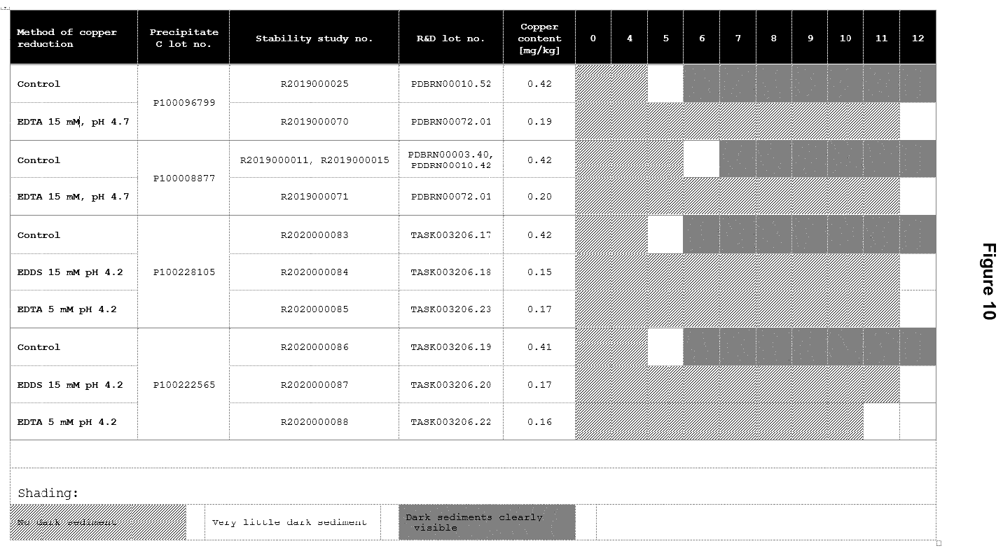

Similar results are shown in FIG. 10, and which correspond to albumin-comprising samples, 12 months after contact with the chelators EDTA or (S,S)-EDDS, with and without acidification. Briefly, albumin samples were obtained by contacting Precipitate C with chelators EDTA (5 mM at pH 4.2 or 15 mM at pH 4.7) or EDDS (15 mM, pH 4.2) during manufacturing of a 25% (w/v) albumin-containing product. Samples were stored for up to 12 months at 40° C., ≤25% relative humidity and the degree of sediment formation was assessed visually and photographs taken.

The visual read-out of the stability studies are summarised and shown in FIG. 10. Samples subjected to chelator treatment show only low levels of dark sediment formation 11-12 months after chelator treatment. In contrast, all samples that are not treated with chelator show clear evidence of dark sediments within about 5 months.

### Example 9: Final Product Formulation

Following the depletion of metal cations using chelators, as described in Examples 2 and 3, the albumin-comprising samples are subjected to downstream manufacturing processes. In the case where the staring material is Precipitate C, the albumin-containing sample is subjected to clarifying depth filtration as described in Example 2, to obtain metal-cation depleted Filtrate D.

The neutralised Filtrate D solution is concentrated to about 130 to 140 g/kg albumin by ultrafiltration, then diafiltered first with at least five times the actual volume of 0.1 to 0.3 M sodium chloride, then with at least 2.5 times the amount of water for injection. By this process the chelator along with any bound metal cations is removed. Also removed is aluminium, other low molecular salts and ethanol.

The albumin solution is then formulated. This is achieved by adjusting the sodium content by adding sodium chloride to a concentration of 140 mmol/L. Then the stabilisers sodium caprylate and sodium N-acetyltryptophanate are added to the albumin solution, corresponding to 20 mmol/L for 25% albumin, 16 mmol/L for 20% albumin and 4 mmol/L for 5% albumin. Water for injection is added to dilute the albumin solutions to the target protein concentration and the pH is adjusted if required by hydrochloric acid or sodium hydroxide solution, respectively.

The final albumin product is sterile filtered prior to being dispensed and then pasteurised.

It will be understood that the invention disclosed and defined in this specification extends to all alternative combinations of two or more of the individual features mentioned or evident from the text or drawings. All of these different combinations constitute various alternative aspects of the invention.

1. A process for preparing a purified albumin composition suitable for pharmaceutical use, from an albumin-comprising sample obtained from blood-derived plasma, the process comprising:
i) providing an albumin-comprising sample obtained from blood-derived plasma;
ii) contacting the albumin-comprising sample with a ligand for binding to or for sequestering metal cations to obtain a metal cation-depleted albumin-comprising sample;
thereby preparing a purified albumin composition.

2. The process of claim 1 wherein the ligand for binding to or for sequestering metal cations is a chelating agent.

3. The process of claim 2, wherein the chelating agent is selected from the group consisting of: ethylenediaminetetraacetic acid (EDTA), ethylene glycol-bis(β-aminoethyl ether)-N,N,N′,N′-tetraacetic acid (EGTA), ethylenediamine-N,N′-disuccinic acid (EDDS), including (S,S)-EDDS, iminodisuccinic acid (IDS), methylglycinediacetic acid (MGDA), triethylenetetramine (Trien), iminodiacetic acid (IDA), nitrilotriacetic acid (NTA), tripolyphosphate (TPP), diethylenetriaminepentaacetic acid (DTPA), sodium diethyldithiocarbamate (DDC), L-Glutamic acid N,N-diacetic acid, tetrasodium salt (GLDA) and penicillamine, or any salt thereof, preferably wherein the chelating agent is a biodegradable chelating agent, such as EDDS, IDS, MGDA and NTA or any isomer or salt thereof.

4. The process of any one of claims 1 to 3, wherein the ligand for binding to or for sequestering metal cations is EDTA, or a salt thereof.

5. The process of any one of claims 1 to 3, wherein the ligand for binding to or for sequestering metal cations is (S,S)-EDDS, or a salt thereof, preferably the trisodium salt thereof.

6. The process of claim 1, wherein the ligand for binding to or for sequestering metal cations is a chelating resin.

7. The process of claim 6, wherein the chelating resin is a cation-exchange resin.

8. The process of claim 7, wherein the cation exchange resin comprises a strong acid exchange group.

9. The process of claim 8, wherein the cation exchange resin comprises a sulfonic acid exchange group.

10. The process of any one of claims 6 to 9, wherein the albumin-comprising sample is contacted with the ligand for binding to or for sequestering metal cations using column chromatography.

11. The process of any one of claims 1 to 10 wherein the ligand for binding to or for sequestering metal cations is a ligand capable of binding to or sequestering copper ions.

12. The process of any one of claims 1 to 11, wherein albumin-comprising sample is selected from: a Cohn Fraction, a Kistler-Nitschmann Fraction or an ammonium-sulfate precipitate obtained from blood-derived plasma.

13. The process of claim 12, wherein the albumin-comprising sample is Cohn Fraction V or Kistler-Nitschmann Precipitate C, or a suspension, filtrate or concentrate thereof.

14. The process of claim 12, wherein the albumin-comprising sample is a filtrate of Fraction V or Precipitate C.

15. The process of any one of claims 1 to 14, wherein process comprises resuspending a precipitate comprising an albumin-comprising sample of blood, prior to contacting the sample with the ligand for binding to or for sequestering metal cations.

16. The process of any one of claims 1 to 15, wherein the albumin-comprising sample is contacted with an amount of chelator that corresponds to at least a 2-fold higher concentration of chelator to copper contained in the sample.

17. The process of claim 16, wherein the albumin-comprising sample is contacted with an amount of chelator that corresponds to at least a 5-fold higher concentration of chelator to copper contained in the sample, preferably at least a 10-fold, at least a 25-fold, at least a 50-fold, at least a 100-fold, at least a 200-fold higher concentration of chelator to copper contained in the sample.

18. The process of any one of claims 1 to 17, wherein the albumin-comprising sample is contacted with a chelator at a concentration of from about 10 μM to about 100 mM chelator, preferably from about 25 μM to about 50 mM chelator, more preferably from about 50 μM to about 15 mM chelator, most preferably from about 50 μM to about 500 μM chelator, especially about 50 μM of chelator.

19. The process of any one of claims 1 to 17, wherein the albumin-comprising sample is contacted with a chelator at a concentration of less than about 100 mM chelator, more preferably less than about 50 mM chelator, or less than about 25 mM chelator, or less than about 15 mM chelator, or less than about 5 mM chelator; most preferably, less than about 1 mM chelator or less than about 500 μM chelator, or less than about 250 μM chelator or less than about 100 μM chelator, especially about 50 μM or less.

20. The process of any one of claims 1 to 19, wherein the process further comprises a step of acidification.

21. The process of claim 20 wherein the acidification occurs before the albumin-comprising sample is contacted with the ligand for binding to or for sequestering metal cations.

22. The process of claim 21, wherein the ligand is a chelating agent, and the step of acidification is performed after the step of contacting the albumin-comprising sample with the chelating agent.

23. The process of claim 21, wherein the ligand is a cation-exchange resin, and the step of acidification is performed prior to contacting the albumin-comprising sample with the cation-exchange resin.

24. The process of any one of claims 20 to 23, wherein the step of acidification comprises contacting the albumin-comprising sample or the albumin-comprising sample depleted of metal cations with an inorganic acid.

25. The process of claim 24 wherein the inorganic acid is selected from the group consisting of: sulphuric acid (H₂SO₄), citric acid (C₆H₈O₇), hydrochloric acid (HCl), phosphoric acid (H₃PO₄), oxalic acid (C₂H₂O₄) and formic acid (CH₂O₂), preferably wherein the inorganic acid is sulphuric acid or hydrochloric acid.

26. The process of any one of claims 20 to 25, wherein the step of acidification results in a reduction in the pH of the albumin-comprising fraction to a pH of approximately 3.0 to 4.5, preferably to a pH of between about 3.5 to about 4.5, or between about 3.6 to 4.4, or between about 3.7 to about 4.3, or between about 3.8 to about 4.2, most preferably between about 3.9 to about 4.2.

27. The process of claim 26 wherein the pH of the albumin-comprising sample that is depleted of metal cations is no lower than about 4.2.

28. The process of any one of claims 20 to 26, wherein the step of acidification results in a reduction in the pH of the albumin-comprising fraction to a pH of approximately 5.6 to 6.0, preferably to a pH of between about 5.8 or about 5.9.

29. The process of any one of claims 1 to 28, further comprising subjecting the albumin-comprising sample that is depleted of metal cations to additional purification steps selected from: a pre-filtration step (e.g., clarifying depth filtration), ultrafiltration (e.g., diafiltration and/or concentration) and combinations thereof.

30. The process of any one of claims 1 to 29, wherein the blood-derived plasma is human blood-derived plasma, optionally wherein the plasma batch size is 1,000-15,000 kg.

31. A purified albumin pharmaceutical composition obtained by the process of any one of claims 1 to 30.

32. A purified albumin pharmaceutical composition, optionally obtained according to by the process of any one of claims 1 to 30, wherein the composition comprises a concentration of copper of no more than about 2.0 μg/g protein, no more than about 1.5 μg/g protein, no more than about 1 μg/g protein, no more than about 0.8 μg/g protein, no more than about 0.5 μg/g protein or no more than about 0.2 μg/g protein; preferably wherein the composition comprises a concentration of copper of no more than about 2.0 μg/g albumin, no more than about 1.5 μg/g albumin, no more than about 1 μg/g albumin, no more than about 0.8 μg/g albumin, no more than about 0.5 μg/g albumin or no more than about 0.2 μg/g albumin.
